SEQUENCE LISTING (12 sequences)

SEQ ID NO: 1 - DNA, 20 bp, Artificial Sequence
  Adapter
aatgatacggcgaccaccga

SEQ ID NO: 2 - DNA, 24 bp, Artificial Sequence
  Adapter
caagcagaagacggcatacgagat

SEQ ID NO: 3 - DNA, 29 bp, Artificial Sequence
  Binding site
tctttccctacacgacgctcttccgatct

SEQ ID NO: 4 - DNA, 30 bp, Artificial Sequence
  Binding site
gtgagtggacttcagtggtgtcagagatgg

SEQ ID NO: 5 - DNA, 33 bp, Artificial Sequence
  Primer
ccucugcugcucucauugucgctcttccgatct

SEQ ID NO: 6 - DNA, 25 bp, Artificial Sequence
  Primer
acacuguacgutagagactccaagc

SEQ ID NO: 7 - DNA, 25 bp, Artificial Sequence
  Primer
acguacagugucgctcttccgatct

SEQ ID NO: 8 - DNA, 25 bp, Artificial Sequence
  Primer
agcucaauccutagagactccaagc

SEQ ID NO: 9 - DNA, 25 bp, Artificial Sequence
  Primer
aggauugagcucgctcttccgatct

SEQ ID NO: 10 - DNA, 25 bp, Artificial Sequence
  Primer
acagacuuacutagagactccaagc

SEQ ID NO: 11 - DNA, 25 bp, Artificial Sequence
  Primer
aguaagucugucgctcttccgatct

SEQ ID NO: 12 - DNA, 33 bp, Artificial Sequence
  Primer
gugcgugcaugauccuacutagagactccaagc

(19) United States
(12) Patent Application Publication
(10) Pub. No.: US 2022/0162589 A1
(43) Pub. Date: May 26, 2022

(54) Analyte Detection Method Employing Concatemers

(71) Applicant: OLINK PROTEOMICS AB, Uppsala (SE)

(72) Inventors: Gowtham Nicklesh KUNDERU, Uppsala (SE); John BROBERG, Uppsala (SE); Martin LUNDBERG, Uppsala (SE); Sara HENRIKSSON, Uppsala (SE)

(21) Appl. No.: 17/534,548

(22) Filed: Nov. 24, 2021

(30) Foreign Application Priority Data
Nov. 25, 2020 (GB) 2018503.9

(51) Int. Cl. C12N 15/10; C12Q 1/6806; C12Q 1/6855; C12Q 1/6876

(52) U.S. Cl. CPC C12N 15/1065; C12Q 1/6806; C12Q 2600/16; C12Q 1/6876; C12Q 1/6855

(57) ABSTRACT

Methods of detecting DNA sequences from multiple pools comprising at least one species of DNA molecule comprise combining the pools to form a combination pool; in the combination pool, generating at least one linear DNA concatemer containing one DNA molecule from each pool, wherein a position of each DNA molecule within the concatemer correlates to the pool from which the DNA molecule originated; and sequencing the concatemers, thereby detecting the DNA sequence of each DNA molecule at each position in each concatemer, wherein each detected DNA sequence is assigned to the pool from which its DNA molecule originated based upon its position within the concatemer.

The sequence listing submitted herewith, entitled “Jan-14-2022-Sequence-Listing.txt”, created Jan. 14, 2022, and having a size of 2432 bytes, is incorporated herein by reference.

### FIELD

The present disclosure and invention provides a method of detecting DNA sequences from multiple pools of DNA molecules. In the method, the pools are combined to form a combination pool, DNA concatemers are generated in the combination pool by joining together a single DNA molecule from each pool in a pre-defined order, and the concatemers are then sequenced. By sequencing each concatemer, multiple DNA sequences are detected, and each DNA sequence detected can be assigned to its pool of origin by its location in the concatemer. The method thereby enables the specific detection of DNA sequences from each of multiple pools. A kit suitable for performing the method is also provided.

### BACKGROUND

Modern proteomics methods require the ability to detect a large number of different proteins (or protein complexes) in a small sample volume. To achieve this, multiplex analysis must be performed. Common methods by which multiplex detection of proteins in a sample may be achieved include proximity extension assays (PEA) and proximity ligation assays (PLA). PEA and PLA are described in WO 01/61037; PEA is further described in WO 03/044231, WO 2004/094456, WO 2005/123963, WO 2006/137932 and WO 2013/113699.

PEA and PLA are proximity assays, which rely on the principle of “proximity probing”. In these methods an analyte is detected by the binding of multiple (generally two) probes, which when brought into proximity by binding to the analyte (hence “proximity probes”) allow a signal to be generated. Typically, the proximity probes each comprise a nucleic acid domain (or moiety) linked to an analyte-binding domain (or moiety) of the probe, and generation of the signal involves an interaction between the nucleic acid moieties. Thus signal generation is dependent on an interaction between the probes (more particularly between their nucleic acid moieties/domains) and hence only occurs when the necessary probes have bound to the analyte, thereby lending improved specificity to the detection system.

In PEA, nucleic acid moieties linked to the analyte-binding domains of a probe pair hybridise to one another when the probes are in close proximity (i.e. when bound to a target), and are then extended using a nucleic acid polymerase. The extension product forms a reporter DNA molecule, detection of which demonstrates the presence in a sample of interest of a particular analyte (the analyte bound by the relevant probe pair). In PLA, nucleic acid moieties linked to the analyte-binding domains of a probe pair come into proximity when the probes of the probe pair bind their target, and may be ligated together, or alternatively they may together template the ligation of separately added oligonucleotides which are able to hybridise to the nucleic acid domains when they are in proximity. The ligation product is then amplified, acting as a reporter DNA molecule. Multiplex analyte detection using PEA or PLA may be achieved by including a unique barcode sequence in the nucleic acid moiety of each probe.

Proximity assays may be used for the detection of any analyte, not just proteins, including nucleic acid analytes, and may be used for multiplex detection of such analytes. Further, other detection assays may also employ nucleic acid reporter molecules, and may be used for the detection of any analyte, for example immunoPCR or immunoRCA assays. A reporter DNA molecule may be provided, or generated during the course of an assay, which comprises a barcode sequence by which it, and thereby its corresponding analyte, may be detected.

A reporter DNA molecule corresponding to a particular analyte may be identified by the barcode sequences it contains. In a multiplex reaction, each reporter DNA molecule may be detected by a technique employed to detect its specific sequence. This may be achieved by sequencing the reporter, or by amplification using specific primers and/or specific detection probes which hybridise to the reporter or its amplicon. For example qPCR may be used to detect reporter molecules of defined sequences, or as described in co-pending application PCT/EP2021/058008, next generation sequencing (NGS) may be used to sequence all reporter DNA molecules generated in a particular assay, thereby identifying all reporter DNA molecules produced. Detection of a particular reporter DNA molecule indicates that the analyte corresponding to that reporter DNA molecule is present in the sample of interest.

In existing methods whereby reporter DNA molecules generated in a detection assay are detected by sequencing, each reporter DNA molecule is individually sequenced and detected. The number of reporter DNA molecules that can be sequenced and detected in any given sequencing reaction is therefore limited by the capacity of the sequencing platform (e.g. flow cell). It would be advantageous to increase the number of reporter DNA molecules that can be detected in an NGS reaction, as this would increase the efficiency of the detection assay.

A method of increasing the throughput of NGS by concatenation of DNA molecules has previously been reported (Schlecht et al., Scientific Reports 7: 5252, 2017), referred to as ConcatSeq. The ConcatSeq technique utilises Gibson Assembly to generate concatemers of DNA molecules of interest, and was reported to increase sequencing throughput more than five-fold. While the production of concatemers for sequencing can increase efficiency per sequencing run, significant limitations still exist for sequencing of complex assays, and particularly for sequencing DNA molecules generated in multiplex detection assays such as PEA and PLA in order to detect the presence of certain analytes in specific samples. It is also often desirable to conduct multiple multiplex detection assays with multiple samples and, again, the number of reporter DNA molecules that can be sequenced and detected in any given sequencing reaction from such multiple multiplex detection assays for analyte identification is limited.

Accordingly, a need exists for further improvements in sequencing efficiency for analysing multiple DNA molecules, and particularly improvements that facilitate DNA sequencing of molecules generated from multiple multiplex detection assays.

### SUMMARY

Accordingly, it is an object of the present invention to provide improvements in sequencing efficiency.

In a first aspect, disclosed and provided herein is a method of detecting DNA sequences from multiple pools. In a first embodiment, the method comprises;

(i) combining the pools to form a combination pool;

(ii) in the combination pool, generating at least one linear DNA concatemer containing one DNA molecule from each pool, wherein a position of each DNA molecule within the concatemer correlates to the pool from which the DNA molecule originated; and

(iii) sequencing the concatemers, thereby detecting the DNA sequence of each DNA molecule at each position in each concatemer, wherein each detected DNA sequence is assigned to the pool from which its DNA molecule originated based upon its position within the concatemer.

In another embodiment, wherein each pool comprises multiple species of DNA molecules, the method comprising:

(i) combining the pools to form a combination pool;

(ii) generating multiple linear DNA concatemers, wherein each concatemer is generated by joining together one random DNA molecule from each pool in a pre-determined order such that the position of each DNA molecule within the concatemer indicates the pool from which it is derived and each concatemer comprises a pre-determined number of DNA molecules; and

(iii) sequencing the concatemers, thereby detecting a DNA sequence from each pool in each concatemer, wherein the DNA sequence from each pool is assigned to that pool based upon its position within its concatemer.

In particular, the pools may comprise DNA molecules which are capable of being concatenated in a pre-defined and directed order. In other words, the DNA molecules in each pool are capable of being concatenated, or linked, only to molecules from a pre-designated, or selected, other pool. Accordingly, each pool is designated, or allocated, a predesignated place or position in the concatemer. The concatemer thus has a pre-determined “pool order” of monomer positions, and the identity of the pool from which each monomer in the concatemer derives may be determined from the position of the monomer in the concatemer. In other words, the position of each DNA molecule within the concatemer correlates to the pool from which it is derived. To allow a concatemer of a predefined order of pools to be constructed, each DNA molecule (i.e. monomer) may be linked to only one (if it is a terminal monomer) or two other DNA molecules (that is to say, each DNA molecule (monomer) may be linked to DNA molecules from only one (if it is a terminal monomer) or two other pools.

Thus, the DNA molecules in a pool may be prepared for concatenation. In an embodiment, the method comprises, prior to step (i), a step of preparing multiple pools of DNA molecules for concatenation, wherein said preparing comprises providing the DNA molecules within each pool with defined end sequences which may be joined in a concatenation step, the DNA molecules in the same pool having the same end sequences and the different pools having different end sequences, such that a DNA molecule from one pool may only be joined to a DNA molecule from one or two pre-determined different pools. A DNA molecule may have one or two end sequences, depending on its position in the conacatemer. Further, a DNA molecule in a terminal position in the concatemer may be provided with a second end sequence for linkage to another molecule (i.e. a molecule which is other than a DNA molecule from a pool), e.g. a sequencing or other adaptor. In one embodiment therefore, the method comprises, prior to combining individual pools, in each pool, joining to each DNA molecule of the pool a first end sequence, and, when the number N of multiple pools is greater than two, for at least N-2 pools, joining to each DNA molecule of each N-2 pool, a second end sequence, wherein each end sequence is different from the other end sequences and each end sequence of each pool is configured to join to one end sequence in one other pool to form the linear DNA concatemers.

In a second aspect, disclosed and provided herein is a kit comprising:

(i) multiple proximity probe pairs, wherein each proximity probe comprises a binding domain specific for an analyte and a nucleic acid domain, and each proximity probe pair is specific for a different analyte, such that on proximal binding of the pair of proximity probes to their respective analyte the nucleic acid domains of the proximity probe pair are capable of interacting to generate a reporter DNA molecule, and wherein in each pair the nucleic acid domain of one proximity probe comprises a first universal primer binding site and a barcode sequence 3′ thereof, and the nucleic acid domain of the other proximity probe comprises a second universal primer binding site and a barcode sequence 3′ thereof;

(ii) a first primer pair, wherein the primers are designed to bind the first and second universal primer binding sites;

(iii) a set of assembly primer pairs suitable for preparing DNA molecules for directed assembly by USER assembly or Gibson assembly into a linear concatemer, wherein each primer comprises, from 5′ to 3′, an assembly site and a hybridisation site, and in each primer pair the hybridisation sites are designed to bind the first and second universal primer binding sites;

(iv) enzymes suitable for assembling DNA fragments by USER assembly or Gibson assembly, wherein the enzymes are suitable for use in the same means of DNA assembly as the assembly primer pairs; and

(v) a second primer pair, wherein each primer comprises a sequencing adaptor, a sequencing primer binding site, an index sequence and a hybridisation site, wherein the hybridisation sites are designed to bind the assembly sites of the assembly primers designed to form the ends of the linear concatemer;

and wherein the first primer in the pair comprises a first sequencing adaptor, a first sequencing primer site and a first index sequence, and the second primer in the pair comprises a second sequencing adaptor, a second sequencing primer site and a second index sequence.

In an embodiment, the proximity probes may be probes for a PEA. In such an embodiment, the proximity probe pair may comprise nucleic acid domains that hybridise to one another and template an extension reaction. Thus, the nucleic acid domain of one proximity probe may prime an extension reaction templated by the nucleic domain of the other probe of the pair. In another embodiment the proximity probes may be probes for a PLA. In such an embodiment, the proximity probe pair comprise nucleic acid domains that hybridise to a common ligation template such that may be ligated together, or nucleic acid domains that template the ligation of one or more added oligonucleotides, and/or prime the amplification of the ligation product.

The methods and kits of the invention are particularly advantageous for sequencing DNA molecules generated in multiple multiplex detection assays. Specifically, the methods and kits make it possible to convey information in relation to the assay based on a particular position in the concatemer, for example in relation to the origin of the sequence which is incorporated into the concatemer at that position. The present invention provides an improved method of generating concatemers for sequencing which is particularly useful in the context of multiplex detection assays such as PEA and PLA, whereby sequencing throughput and efficiency are increased by concatenating reporter DNA molecules from multiple pools (i.e., resulting from multiple multiples assays) in a predefined order, such that the location of each reporter DNA sequence within the resultant concatemers is indicative of the pool (assay) from which it originates. Each pool may be generated, for instance, from a separate sample, or using a separate panel of proximity probes. The method is particularly advantageous when each pool of reporter DNA molecules is generated using probes carrying the same set of nucleic acid moieties. The ability to assign each reporter DNA sequence in a concatemer to a particular pool of origin means that identical reporter sequences present within multiple pools can be distinguished based on their locations within the concatemers.

The methods and kits provided herein thus have particular utility in the context of proximity assays (e.g. PEA and PLA assays), but their utility and advantages are not limited to these assays. The methods and kits of the invention can be used in any context where it is desired to analyse a pool of DNA molecules.

### DETAILED DESCRIPTION

As mentioned above, the first aspect provides a method of detecting DNA sequences from multiple pools. The DNA sequences are detected by DNA sequencing. A given DNA sequence is identified by sequencing and thus its presence in a pool is confirmed.

A “pool” as used herein is a mixture (e.g. a solution) containing at least one, but typically multiple, species of DNA molecules. A “species” of DNA molecule means herein a DNA molecule with a particular sequence. Each pool therefore typically comprises multiple, or in other words a plurality of, different DNA molecules (i.e. DNA molecules having different sequences). By “multiple” or “plurality” as used herein is meant at least two. A pool comprising a plurality of different DNA molecules may be prepared or generated in any convenient or desired way. Different nucleic acid molecules may occur naturally in a sample, and different samples may represent different pools, Alternatively, pools may be prepared by mixing nucleic acid molecules. A pool of nucleic acid molecules may be generated, for example a pool of reporter nucleic acid molecules may be generated by a multiplex assay detecting multiple different analytes in a sample, as discussed further below. Thus each pool comprises at least two species of DNA molecules, e.g. at least 10, at least 50 or at least 100 or more species of DNA molecules. Multiple copies of each species of DNA molecule may be present in the respective pools. The DNA sequences from each pool detected in the method are the sequences of, or sequences comprised within, the various species of DNA molecules present in the pools. The sequences detected may be the entirety of each DNA molecule, or may be parts of each DNA molecule (i.e. the sequences detected may be located within each DNA molecule), as discussed further below.

Each pool may comprise the same number of species of DNA molecule, or each pool may comprise a different number of species of DNA molecule. Each pool may comprise similar concentrations of each DNA molecule, or different concentrations. It is preferred that the total number of DNA molecules within each pool are similar.

The term “DNA molecule” as used herein has its standard meaning in the art, i.e. a polymer of deoxyribonucleotides. Each DNA molecule may be single- or double-stranded, though generally will be double-stranded. Generally, the DNA molecules will comprise (or primarily comprise) the four standard DNA bases (adenine, thymine, cytosine and guanine), but may also comprise other non-standard DNA bases, e.g. modified bases and DNA adducts. As described further below, in a particular embodiment the DNA molecules may comprise uracil bases. The DNA molecules in the pools are linear. Circular DNA molecules must be linearised in order for concatenation to take place.

The method is used to detect DNA sequences from a plurality of pools, that is to say at least 2 pools. Preferably in one embodiment, the method is used to detect DNA sequences from at least 3 pools, e.g. 3, 4, 5, 6, 7 or 8 pools or more. In particular embodiments the method is used to detect sequences from 3 to 8 pools, 3 to 7 pools, 3 to 6 pools, or 4 to 6 pools. In practice there is no real limit on the length of the concatemer, and hence on the number of pools, and this could be much higher, if desired.

In step (i), the pools of DNA molecules are combined to form a combination pool. That is to say, all the pools are added together and mixed to form a single reaction mixture The reaction mixture thus comprises the DNA molecules from each pool.

Following combination (i.e. mixing) of the pools, a concatenation reaction is performed in the combination pool. The concatenation reaction generates multiple linear DNA concatemers from the pooled DNA molecules. In general parlance, a DNA concatemer is a molecule containing linked copies of a repeating DNA unit. The same is true in the claimed method, in that the repeating DNA units are the DNA molecules from the pools. As further discussed below, each DNA molecule generally has a common structure (and some may share a common sequence), which is thus repeated along the concatemer. It will be understood, however, that the repeating unit, that is the monomer of the concatemer, need not be identical. The monomers of the concatemer are constituted by the individual DNA molecules, one from each pool, that are linked together in the concatemer. The concatemers generated are linear, i.e. they are not circular molecules but rather have two ends.

Each concatemer is generated by joining together one DNA molecule from each pool. Thus, if e.g. the method is being performed on 4 pools of DNA molecules, the resulting concatemers will each comprise 4 repeated units, i.e. one DNA molecule from each of the 4 pools. The concatemers generated therefore comprise a pre-determined number of DNA molecules (corresponding to the number of pools) and have a pre-defined length, correlated to the number of pools used in the method. Although each concatemer comprises one DNA molecule from each pool, the specific DNA molecule from each pool incorporated into each concatemer is random, i.e. each concatemer comprises a single DNA molecule from each pool, and the DNA molecules from each pool assembled into each concatemer are selected at random.

As noted above, when the pools have multiple DNA molecules, multiple concatemers are generated in the method. The number of concatemers generated corresponds to the total number of DNA molecules in each pool (and in particular to the total number of DNA molecules in the pool with the smallest number of total DNA molecules—as mentioned above it is preferred that the pools contain similar numbers of DNA molecules). It is preferred that the concatenation reaction essentially exhausts the combined DNA molecules, such that essentially all the DNA molecules from the pools are incorporated into concatemers.

During concatenation, the DNA molecules from each pool are assembled in a pre-defined order, such that the location of each DNA molecule within each concatemer (or in other words its position in the concatemer) is defined based on the pool from which the DNA molecule originates. In each concatemer formed, the DNA molecules are arranged in the same order (based on the pools from which each DNA molecule originates). Thus there is an order of pools (a so-called “pool order”) which is pre-defined, and is the same for each concatemer. Any suitable method may be used to perform concatenation. The sole requirement is that the method is suitable for performing directed assembly of DNA molecules.

The fact that each concatemer comprises a DNA molecule from each pool, with the DNA molecules arranged in a pre-defined order based on their pool of origin means that upon sequencing of each concatemer, the pool of origin of each DNA molecule within the concatemer can be determined simply based on the position of the DNA molecule within the concatemer. For example, if the method is performed on 4 pools, Pools A, B, C and D, each pool will be pre-assigned to a location in the concatemer. For instance, Pool A may be assigned position 1, Pool B position 2, Pool C position 3 and Pool D position 4. Each concatemer will thus contain four DNA molecules assembled in the following order:
Pool A Molecule—Pool B Molecule—Pool C Molecule—Pool D Molecule
Sample A Molecule—Sample B Molecule—Sample C Molecule—Sample D Molecule

This is depicted schematically in FIG. 7, which will be discussed in further detail below and which shows how a molecule from each of 4 pools, A, B, C, and D, is incorporated into a concatemer. The figure depicts a single molecule generated in each pool.

Since DNA is double-stranded, and each strand can be read separately upon sequencing, clearly the DNA molecules will be arranged in opposing orders in the two strands. Thus in the above example, if the above order is the order of the molecules in the first strand of the concatemer, the second strand of the concatemer will contain the four DNA molecules in the reverse order, i.e.:
Pool D Molecule—Pool C Molecule—Pool B Molecule—Pool A Molecule

The two strands of each concatemer are distinguishable. Generally when the method is performed the possible sequences of the DNA molecules within each pool are known, e.g. the sequences of DNA molecules within each pool are selected from a known set of DNA sequences, such that each DNA molecule can only have one of a limited set of DNA sequences. In this embodiment, the two strands can be distinguished based on whether they comprise the forward or reverse sequences of each DNA molecule. Thus, in the example above, the first strand comprises the forward sequences of each DNA molecule and the reverse strand comprises the reverse sequence of each DNA molecule (by reverse here is of course meant the reverse complement). It is thus possible to determine whether each strand, when sequenced, is the forward or reverse strand of a concatemer, and thereby establish the pool of origin of each DNA molecule within the concatemer. To this end, it may be preferred if the DNA molecules do not have palindromic sequences.

Alternatively or additionally, and particularly if the possible sequences of the DNA molecules are not known, the ends of each concatemer may be tagged so that they can be distinguished. In particular, a terminus-specific tag may be added to one or both ends of the concatemer. A first terminus-specific tag can be attached to one end of each DNA concatemer, e.g. the free end of the DNA molecule at position 1. Optionally a second terminus-specific tag can be attached to the free end of the DNA molecule at the other end of the concatemer (e.g. in the example above, the second tag would be attached to the free end of the DNA molecule at position 4). The terminus specific tags enable orientation of each concatemer sequence even if this is not possible from the sequences of the DNA molecules contained within it. Where two terminus-specific tags are used, the first and second terminus-specific tags have different sequences. Examples of suitable tags are described below, for instance a sequencing primer binding site may act as a terminus-specific tag.

Once the concatemers have been generated, they are sequenced. Any suitable sequencing method may be used, as discussed further below. Once the concatemers have been sequenced, the DNA molecules within each concatemer can be identified. This means that the DNA sequence from each pool within each concatemer is detected. Since the pool of origin of each DNA sequence can be determined by the location of the sequence within each concatemer, this allows each DNA sequence to be assigned to its pool of origin based on its position within its concatemer. By sequencing all concatemers, all the DNA sequences present in each pool can be identified.

Commonly, the method comprises a preparation step, performed prior to step (i). In the preparation step, the multiple pools of DNA molecules are prepared for concatenation by providing the DNA molecules within each pool with defined end sequences which can be joined in the concatenation step. Typically, each DNA molecule will receive two end sequences, one at each end, although this is not strictly necessary, and DNA molecules designated as a terminal monomer in the concatemer may receive only one, In the preparation step, all the DNA molecules within each pool are provided with the same end sequences (though in each pool, the two end sequences are not the same—each DNA molecule is provided with two different end sequences). However, different end sequences are provided to the DNA molecules in each different pool. That is to say, that within each pool all DNA molecules are provided with the same pair of end sequences, but the DNA molecules from each different pool are provided with a different pair of end sequences. Said another way, each DNA molecule of a pool is provided with a first end sequence, and, when the number N of multiple pools is greater than two, for at least N-2 pools, each DNA molecule of each N-2 pool is provided with a second end sequence, wherein each end sequence is different from the other end sequences and each end sequence of each pool is configured to join to one end sequence in one other pool to form the linear DNA concatemers. As noted, the two DNA molecules that will be at the termini of a concatemer are not required to have an end sequence at their end positioned at a terminus of the concatemer.

By end sequences, here, is meant sequences which are attached to the ends of the DNA molecules in each pool, such that following their attachment, the defined end sequences form both ends of each DNA molecule within the pool. Thus each DNA molecule is provided with a first defined end sequence which is attached to one end of the DNA molecule, and a second defined end sequence which is attached to the other end of the DNA molecule. As specified above, the first and second end sequences are different. An end sequence may alternatively be referred to as an adaptor sequence, more particularly a terminal adaptor sequence or an assembly adaptor sequence.

The end sequences are configured to enable the joining of the DNA molecules in the various pools to one another in a defined order. Thus each end sequence (aside from those designed to form the termini of the concatemer) has a paired end sequence (e.g. a complementary end sequence) within the set of end sequences used. For each pair of end sequences, the two end sequences are provided to different pools. That is to say, of a given pair of end sequences, the first end sequence is attached to the DNA molecules in a first pool and the second end sequence is attached to the DNA molecules in a second pool. This means that following combination of the pools, DNA molecules from the first pool can be joined to DNA molecules from the second pool via their paired end sequences. Thus in the concatenation reaction, across all pools, via their paired end sequences, the DNA molecules from each pool can be joined to the DNA molecules from two other, defined pools (with the exception of the DNA molecules designed to form the termini of the concatemer, which are each only joined to one other DNA molecule), in a defined orientation. Suitable types of paired end sequences are known in the art, for instance each pair of end sequences may share a specific restriction site that can be used to join them. Other means for directed joining of DNA molecules are discussed below.

As discussed further below, the end sequences can be added to the ends of the DNA molecules in the pools by any suitable method. Amplification using primers containing the end sequences is a preferred method, e.g. amplification by PCR.

Thus in a particular embodiment, provided herein is a method of detecting DNA sequences from multiple pools, wherein each pool comprises multiple species of DNA molecule, the method comprising:

(i) preparing the DNA molecules within each pool for concatenation, by providing the DNA molecules within each pool with defined end sequences which may be joined in a concatenation step, the DNA molecules in the same pool having the same end sequences and the different pools having different end sequences, such that a DNA molecule from one pool may only be joined to a DNA molecule from one or two pre-determined different pools;

(ii) combining the pools;

(iii) generating multiple linear DNA concatemers of a pre-defined length, wherein each concatemer is generated by joining together one random DNA molecule from each pool in a pre-determined order such that the position of each DNA molecule within the concatemer indicates the pool from which it is derived and each concatemer comprises a pre-determined number of DNA molecules; and

(iv) sequencing the concatemers, thereby detecting a DNA sequence from each pool in each concatemer, wherein the DNA sequence from each pool is assigned to that pool based upon its position within its concatemer.

In a particular embodiment, the DNA molecules to be concatenated and sequenced in the method are amplicons generated in a DNA amplification reaction. The amplicon may be generated by any known DNA amplification reaction, e.g. LAMP (loop-mediated isothermal amplification) but most preferably is generated by PCR.

In other words, prior to concatenation, the DNA molecules may be generated by an amplification reaction (preferably PCR). The DNA molecules in each pool are, in this instance, generated by a separate amplification reaction, e.g. by separate PCRs. The same PCR may be used both to generate the DNA molecules in the pools, and also to add end sequences to them as described above. In this embodiment, the end sequences are included at the 5′ termini of the primers used for the amplification (or at least 5′ to the primers' hybridisation sites). In an alternative embodiment, a first PCR is performed in each pool to generate the DNA molecules, and subsequently a second PCR is performed in each pool to add end sequences to the DNA molecules. See, for example, FIG. 7, which shows PCR1 performed in each pool to generate the DNA molecules, and subsequently PCR2 performed in each pool to add end sequences to the DNA molecules.

In a particular embodiment, each DNA molecule is a reporter DNA molecule specific for an analyte (as used herein, the terms “reporter DNA” and “reporter DNA molecule” are interchangeable). The term “analyte” as used herein means any substance (e.g. molecule) or entity it is desired to detect using a detection assay. In this embodiment, the method of the invention (as described above) constitutes a part of the detection assay. The analyte is thus the or a “target” of a detection assay.

The analyte may accordingly be any biomolecule or chemical compound it is desired to detect, for example a peptide or protein, or a nucleic acid molecule or a small molecule, including organic and inorganic molecules. The analyte may be a cell or a microorganism, including a virus, or a fragment or product thereof. It will be seen therefore that the analyte can be any substance or entity for which a specific binding partner (e.g. an affinity binding partner) can be developed. All that is required is that the analyte is capable of simultaneously binding at least two binding partners (more particularly, the analyte-binding domains of at least two proximity probes).

As detailed above, the method has particular utility in a proximity probe-based assay. Such assays have found particular utility in the detection of proteins or polypeptides. Analytes of particular interest thus include proteinaceous molecules such as peptides, polypeptides, proteins or prions or any molecule which includes a protein or polypeptide component, etc., or fragments thereof. In a particular embodiment the analyte is a wholly or partially proteinaceous molecule, most particularly a protein. That is to say, in an embodiment the analyte is or comprises a protein. In this context, the term “protein” is used to include any peptide or polypeptide.

The analyte may be a single molecule or a complex molecule that contains two or more molecular subunits, which may or may not be covalently bound to one another, and which may be the same or different. Thus in addition to cells or microorganisms, such a complex analyte may also be a protein complex, or a biomolecular complex comprising a protein and one or more other types of biomolecule. Such a complex may thus be a homo- or hetero-multimer. Aggregates of molecules (e.g. proteins) may also constitute target analytes, for example aggregates of the same protein or different proteins. The analyte may also be a complex between proteins or peptides and nucleic acid molecules such as DNA or RNA. Of particular interest may be the interactions between proteins and nucleic acids, e.g. regulatory factors, such as transcription factors, and DNA or RNA. Thus in a particular embodiment the analyte is a protein-nucleic acid complex (e.g. a protein-DNA complex or a protein-RNA complex). In another embodiment, the analyte is a non-nucleic acid analyte, by which is meant an analyte which does not comprise a nucleic acid molecule. Non-nucleic acid analytes include proteins and protein complexes, as mentioned above, small molecules and lipids.

As noted above, each DNA molecule may be a reporter DNA molecule for an analyte. In this embodiment, the detection assay is used for detection of one or more analytes in a sample. In one embodiment, the presence of a particular analyte in the sample results in the production during the detection assay of a nucleic acid molecule with a particular nucleotide sequence, which is known to correspond to the particular analyte. In another embodiment, a nucleic acid molecule with a particular nucleotide sequence may be provided in the assay as a reporter for the presence of the analyte, e.g. as a tag or label for a moiety which binds to the analyte. Detection of the particular nucleotide sequence indicates that the analyte to which the sequence corresponds is present in the sample. A “reporter DNA molecule” is thus a nucleic acid molecule whose presence (or detection) or generation during the detection assay indicates the presence in the sample of a particular analyte. In an embodiment, each pool comprises the reporter DNA molecules generated in a separate detection assay. For example, if three detection assays are performed, three pools of reporter DNA molecules may be generated.

A detection assay may be performed in simplex, where each assay detects a particular analyte in a sample, or in multiplex, wherein the assay detects multiple different analytes in the sample. Reporter DNA molecules from multiple simplex assays may be pooled to create a pool comprising multiple different reporter molecules. Alternatively, a multiplex assay may yield a pool of different reporter molecules. For example, a multiplex assay may be performed on a single sample to detect multiple different analytes. Multiple pools may be generated from multiple multiplex assays, wherein each multiplex assay yields a different pool.

As noted above, each reporter DNA molecule is specific for a particular analyte. Thus, a reporter DNA molecule identifies a given analyte, or more particularly, may contain a sequence or domain which functions as a barcode sequence, by which an analyte may be detected. Broadly speaking, a barcode sequence may be defined as a nucleotide sequence within the reporter DNA molecule which identifies the reporter, and thus the detected analyte. It may be that the entirety of each reporter DNA molecule generated in the detection assays is unique, in which case the entire reporter DNA molecule may be considered a barcode sequence. More commonly, one or more smaller sections of the reporter DNA molecule act as barcode sequences.

Thus in a particular embodiment, there is provided a method for detecting analytes in one or more samples, the method comprising:

(i) performing multiple separate detection assays, wherein each detection assay generates a pool of multiple different reporter DNA molecules, each of which is specific for a particular analyte;

(ii) combining the pools;

(iii) generating multiple linear DNA concatemers of a pre-defined length, wherein each concatemer is generated by joining together one random reporter DNA molecule from each pool in a pre-determined order such that the position of each reporter DNA molecule within the concatemer indicates the pool from which it is derived and each concatemer comprises a pre-determined number of reporter DNA molecules; and

(iv) sequencing the concatemers, thereby detecting a reporter DNA sequence from each pool in each concatemer, wherein the reporter DNA sequence from each pool is assigned to that pool based upon its position within its concatemer, and thereby detecting the analytes in the or each sample.

In particular, the method may comprise after step (i) a step of providing the reporter DNA molecules within each pool with defined end sequences which may be joined in a concatenation step, the reporter DNA molecules in the same pool all having the same end sequences and the different pools having different end sequences, such that a reporter DNA molecule from one pool may only be joined to a reporter DNA molecule from one or two pre-determined different pools;

In this embodiment it is preferred that the multiple detection assays are all the same (i.e. the same assay is used to generate each pool of reporter DNA molecules).

The term “detecting” or “detected” is used broadly herein to mean determining the presence or absence of an analyte (i.e. determining whether a target analyte is present in a sample of interest or not). Accordingly, if this embodiment of the invention is performed and an attempt is made to detect a particular analyte of interest in a sample, but the analyte is not detected because it is not present in the sample, the step of “detecting the analyte” has still been performed, because its presence or absence from the sample has been assessed. The step of “detecting” an analyte is not dependent on that detection proving successful, i.e. on the analyte actually being detected.

Detecting an analyte may further include any form of measurement of the concentration or abundance of the analyte in the sample. Either the absolute concentration of a target analyte may be determined, or a relative concentration of the analyte, for which purpose the concentration of the target analyte may be compared to the concentration of another target analyte (or other target analytes) in the sample or in other samples. Thus “detecting” may include determining, measuring, assessing or assaying the presence or absence or amount of an analyte. Quantitative and qualitative determinations, measurements or assessments are included, including semi-quantitative determinations. Such determinations, measurements or assessments may be relative, for example when two or more different analytes in a sample are being detected, or absolute. As such, the term “quantifying” when used in the context of quantifying a target analyte in a sample can refer to absolute or to relative quantification. Absolute quantification may be accomplished by inclusion of known concentration(s) of one or more control analytes and/or referencing the detected level of the target analyte with known control analytes (e.g. through generation of a standard curve). Alternatively, relative quantification can be accomplished by comparison of detected levels or amounts between two or more different target analytes to provide a relative quantification of each of the two or more different analytes, i.e. relative to each other. Methods by which quantification can be achieved in the method of the invention are discussed further below.

The methods of the invention are particularly advantageous for detecting analytes in one or more samples. As detailed above, each separate detection assay may be performed on a different sample. In this case, each detection assay may be performed in order to detect the same analytes in multiple different samples, or to detect different analytes in different samples. Alternatively, each detection assay may be performed on the same sample, with different analytes detected in each separate detection assay. Alternatively, a combination may be used, with multiple samples assayed, and multiple separate detection assays performed for each of the multiple samples.

Any sample of interest may be assayed according to the method (i.e. according to all embodiments of the method). That is to say any sample which contains or may contain analytes of interest, and which a person wishes to analyse to determine whether or not it contains analytes of interest, and/or to determine the concentrations of analytes of interest therein.

Any biological or clinical sample may thus be analysed, e.g. any cell or tissue sample of or from an organism, or a body fluid or preparation derived therefrom, as well as samples such as cell cultures, cell preparations, cell lysates etc. Environmental samples, e.g. soil and water samples, or food samples may also be analysed according to the method herein. The samples may be freshly prepared or they may be prior-treated in any convenient way, e.g. for storage.

Representative samples thus include any material which may contain a biomolecule, or any other desired or target analyte, including for example foods and allied products, clinical and environmental samples. The sample may be a biological sample, which may contain any viral or cellular material, including prokaryotic or eukaryotic cells, viruses, bacteriophages, mycoplasmas, protoplasts and organelles. Such biological material may thus comprise any type of mammalian and/or non-mammalian animal cell, plant cells, algae including blue-green algae, fungi, bacteria, protozoa etc. It may further be a prepared or synthetic sample, for example a sample containing isolated or purified analytes.

The sample may be a clinical sample, for instance whole blood and blood-derived products such as plasma, serum, buffy coat and blood cells, urine, faeces, cerebrospinal fluid or any other body fluid (e.g. respiratory secretions, saliva, milk etc.), tissues and biopsies. In an embodiment the sample is a plasma or serum sample. Thus the method may be used in the detection of biomarkers, for instance, or to assay a sample for pathogen-derived analytes or analytes associated with a disease or clinical condition. The sample may in particular be derived from a human, though the method may equally be applied to samples derived from non-human animals (i.e. veterinary samples). The sample may be pre-treated in any convenient or desired way to prepare it for use in the method, for example by cell lysis or removal, etc.

In one embodiment of the analyte detection method each of the multiple separate detection assays is used to detect multiple analytes. In other words in an embodiment each detection assay is a multiplex detection assay.

As used herein, the term “multiplex” is used to refer to an assay in which multiple (i.e. at least two) different detection assays are performed at the same time, in the same reaction vessel or reaction mixture. For example, multiple different analytes are assayed at the same time. Preferably each multiplex detection assay is used to detect at least 5, 10, 20, 50, 100, 150 200, 250 or 300 analytes. Thus, in an embodiment, the reporter DNA molecules are generated by a multiplex detection assay performed on a sample, and the method comprises performing multiple multiplex detection assays on one or more samples, in order to detect multiple analytes in each sample, and each multiplex detection assay yields a pool of reporter DNA molecules.

Thus in a particular embodiment, there is provided a method for detecting multiple analytes in one or more samples, the method comprising:

(i) performing multiple separate multiplex detection assays, wherein each multiplex detection assay detects multiple analytes in a sample, and each multiplex detection assay generates a pool of reporter DNA molecules, each of which is specific for a particular analyte;

(ii) combining the pools;

(iii) generating multiple linear DNA concatemers of a pre-defined length, wherein each concatemer is generated by joining together one random reporter DNA molecule from each pool in a pre-determined order such that the position of each reporter DNA molecule within the concatemer indicates or correlates to the pool from which it is derived and each concatemer comprises a pre-determined number of reporter DNA molecules; and

(iv) sequencing the concatemers, thereby detecting a reporter DNA sequence from each pool in each concatemer, wherein the reporter DNA sequence from each pool is assigned to that pool based upon its position within its concatemer, and thereby detecting the analytes in the or each sample.

In particular, the method may comprise after step (i) of performing multiple separate multiplex detection assays, a step of providing the reporter DNA molecules within each pool with defined end sequences which may be joined in a concatenation step, the reporter DNA molecules in the same pool all having the same end sequences and the different pools having different end sequences, such that a reporter DNA molecule from one pool may only be joined to a reporter DNA molecule from one or two pre-determined different pools;

As detailed above, it is preferred that each multiplex detection assay is the same (i.e. the same assay is used to generate each pool of reporter DNA molecules). Also as detailed above, each multiplex detection assay may be performed on a different sample. In this case, each multiplex detection assay may be performed in order to detect the same analytes in multiple different samples, or to detect different analytes in different samples. Alternatively, each multiplex detection assay may be performed on the same sample, with different analytes detected in each separate multiplex detection assay. Alternatively, a combination may be used, with multiple samples assayed, and multiple separate multiplex detection assays performed for each of the multiple samples.

The detection assays and multiplex detection assays described above may utilise PCR to generate the reporter DNA molecules to be detected. In a particular embodiment, a first PCR is performed in the detection assays and multiplex detection assays, and subsequently a second PCR is performed. In such an embodiment the first PCR, PCR1 in FIG. 7, may generate a first PCR product, and the first PCR products may then be modified by a second PCR, PCR2 in FIG. 7, in order to prepare the first PCR products for concatenation. In this embodiment the second PCR generates the pools of DNA molecules. That is to say, the second PCR generates the DNA molecules that are subsequently combined and concatenated. In this embodiment the second PCR is used to provide the products of the first PCR with defined end sequences to be joined in the concatenation step, as described above. Both the first and second PCR reactions are therefore performed before the pools are combined.

In particular embodiments, the detection assays and multiplex detection assays described above are proximity probe-based detection assays, e.g. PLAs or PEAs. In a representative embodiment each detection assay is a proximity extension assay (PEA). Similarly each multiplex detection assay may be a proximity extension assay (i.e. a multiplex proximity extension assay).

Proximity extension assays (PEAs) are briefly described above. As noted above, both of these techniques rely on the use of pairs of proximity probes. PEAs are generally discussed in WO 2012/104261 which is incorporated herein by reference.

A proximity probe is defined herein as an entity comprising a binding domain specific for an analyte (or alternatively expressed an “analyte-specific binding domain”), and a nucleic acid domain. By “specific for an analyte” or “analyte-specific” is meant that the analyte-binding domain directly or indirectly specifically recognises and binds a particular target analyte, i.e. it binds its target analyte with higher affinity than it binds to other analytes or moieties. The binding domain may bind directly to the analyte, i.e. it may be a primary binding partner therefor, or it may bind indirectly to the analyte, i.e. it may be a secondary binding partner therefor. In the latter case, the binding domain may bind to a primary binding partner for the analyte. In an embodiment, the binding domain is an antibody, or a fragment or derivative of an antibody which contains an antigen-binding domain, in particular wherein the antibody is a monoclonal antibody Examples of such antibody fragments or derivatives include Fab, Fab′, F(ab′)₂ and scFv molecules.

The nucleic acid domain of a proximity probe may be a DNA domain or an RNA domain. Preferably it is a DNA domain. The nucleic acid domains of the proximity probes in each pair typically are designed to hybridise to one another, or to one or more common oligonucleotide molecules (to which the nucleic acid domains of both proximity probes of a pair may hybridise). Accordingly, the nucleic acid domains must be at least partially single-stranded. In certain embodiments the nucleic acid domains of the proximity probes are wholly single-stranded. In other embodiments, the nucleic acid domains of the proximity probes are partially single-stranded, comprising both a single-stranded part and a double-stranded part.

Proximity probes are typically provided in pairs, each pair specific for a target analyte. By this is meant that within each proximity probe pair, both probes comprise binding domains specific for the same analyte. In a multiplex detection assay multiple different probe pairs are used in each detection assay, each probe pair being specific for a different analyte. That is to say, the analyte-binding domains of each different probe pair are specific for a different target analyte.

The nucleic acid domains of each proximity probe are designed dependent on the method in which the probes are to be used. A representative sample of proximity extension assay formats is shown schematically in FIG. 1 and these embodiments are described in detail below. In general, in a proximity extension assay, upon binding of a pair of proximity probes to their target analyte the nucleic acid domains of the two probes come into proximity of each other and interact (i.e. directly or indirectly hybridise to one another). The interaction between the two nucleic acid domains yields a nucleic acid duplex comprising at least one free 3′ end (i.e. at least one of the nucleic acid domains within the duplex has a 3′ end which can be extended). Addition or activation of a nucleic acid polymerase enzyme within the assay mix leads to extension of the at least one free 3′ end. Thus at least one of the nucleic acid domains within the duplex is extended, using its paired nucleic acid domain as template. The extension product obtained is a reporter nucleic acid molecule as used herein, comprising a barcode sequence which indicates the presence of the analyte bound by the proximity probe pair from which the extension product was produced. In particular, the barcode sequence of the reporter molecule may comprise a barcode sequence from the nucleic acid domain of each probe in the pair. That is, each nucleic acid domain of the proximity probe pair contributes to the barcode sequence of the reporter molecule, or in other words may be seen to contain a partial barcode sequence.

Version 1 of FIG. 1 depicts a “conventional” proximity extension assay, wherein the nucleic acid domain (shown as an arrow) of each proximity probe is single-stranded and is attached to the analyte-binding domain (shown as an inverted “Y”) by its 5′ end, thereby leaving two free 3′ ends. When said proximity probes bind to their respective analyte (the analyte is not shown in the figure) the nucleic acid domains of the probes, which are complementary at their 3′ ends, are able to interact by hybridisation, i.e. to form a duplex. The addition or activation of a nucleic acid polymerase enzyme in the assay mixture allows each nucleic acid domain to be extended using the nucleic acid domain of the other proximity probe as template. The resultant extension product is a reporter nucleic acid molecule which is detected, thereby detecting the analyte bound by the probe pair.

Version 2 of FIG. 1 depicts an alternative proximity extension assay, wherein the nucleic acid domain of the first proximity probe is attached to the analyte-binding domain by its 5′ end and the nucleic acid domain of the second proximity probe is attached to the analyte-binding domain by its 3′ end. The nucleic acid domain of the second proximity probe therefore has a free 5′ end (shown as a blunt arrow), which cannot be extended. The 3′ end of the second proximity probe is effectively “blocked”, i.e. it is not “free” and it cannot be extended because it is conjugated to, and therefore blocked by, the analyte-binding domain. In contrast to version 1, only the nucleic acid domain of the first proximity probe (which has a free 3′ end) may be extended using the nucleic acid domain of the second proximity probe as a template, yielding an extension product (i.e. reporter nucleic acid molecule).

In version 3 of FIG. 1, like version 2, the nucleic acid domain of the first proximity probe is attached to the analyte-binding domain by its 5′ end and the nucleic acid domain of the second proximity probe is attached to the analyte-binding domain by its 3′ end. The nucleic acid domain of the second proximity probe therefore has a free 5′ end (shown as a blunt arrow), which cannot be extended. However, in this embodiment, the nucleic acid domains which are attached to the analyte binding domains of the respective proximity probes do not have regions of complementarity and therefore are unable to form a duplex directly. Instead, a third nucleic acid molecule is provided that has a region of homology with the nucleic acid domain of each proximity probe. This third nucleic acid molecule acts as a “molecular bridge” or a “splint” between the nucleic acid domains. This “splint” oligonucleotide bridges the gap between the nucleic acid domains, allowing them to interact with each other indirectly, i.e. each nucleic acid domain forms a duplex with the splint oligonucleotide.

Thus, when the proximity probes bind to their respective analyte-binding targets on the analyte, the nucleic acid domains of the probes each interact by hybridisation, i.e. form a duplex, with the splint oligonucleotide. It can be seen therefore that the third nucleic acid molecule or splint may be regarded as the second strand of a partially double stranded nucleic acid domain provided on one of the proximity probes. In this embodiment the nucleic acid domain of the first proximity probe (which has a free 3′ end) may be extended using the “splint oligonucleotide” (or single stranded 3′ terminal region of the other nucleic acid domain) as a template. Alternatively or additionally, the free 3′ end of the splint oligonucleotide (i.e. the unattached strand, or the 3′ single-stranded region) may be extended using the nucleic acid domain of the first proximity probe as a template.

In one embodiment, the splint oligonucleotide may be provided as a separate component of the assay. In other words it may be added separately to the reaction mix (i.e. added separately to the proximity probes to the sample containing the analytes). It may nonetheless be regarded as a strand of a partially double-stranded nucleic acid domain, albeit that it is added separately. Alternatively, the splint may be pre-hybridised to one of the nucleic acid domains of the proximity probes, i.e. hybridised prior to contacting the proximity probe with the sample. In this embodiment, the splint oligonucleotide can be seen directly as part of the nucleic acid domain of the proximity probe.

Hence, the extension of the nucleic acid domain of the proximity probes as defined herein encompasses also the extension of the “splint” oligonucleotide. Advantageously, when the extension product arises from extension of the splint oligonucleotide, the resultant extended nucleic acid strand is coupled to the proximity probe pair only by the interaction between the two strands of the nucleic acid molecule (by hybridisation between the two nucleic acid strands). Hence, in these embodiments, the extension product may be dissociated from the proximity probe pair using denaturing conditions, e.g. increasing the temperature, decreasing the salt concentration etc.

Version 4 of FIG. 1 is a modification of Version 1, wherein the nucleic acid domain of the first proximity probe comprises at its 3′ end a sequence that is not fully complementary to the nucleic acid domain of the second proximity probe. Thus, when said proximity probes bind to their respective analyte the nucleic acid domains of the probes are able to interact by hybridisation, i.e. to form a duplex, but the extreme 3′ end of the nucleic acid domain (the part of the nucleic acid molecule comprising the free 3′ hydroxyl group) of the first proximity probe is unable to hybridise to the nucleic acid domain of the second proximity probe and therefore exists as a single stranded, unhybridised, “flap”. On the addition or activation of a nucleic acid polymerase enzyme, only the nucleic acid domain of the second proximity probe may be extended using the nucleic acid domain of the first proximity probe as template.

Version 5 of FIG. 1 could be viewed as a modification of Version 3. However, in contrast to Version 3, the nucleic acid domains of both proximity probes are attached to their respective analyte-binding domains by their 5′ ends. In this embodiment the 3′ ends of the nucleic acid domains are not complementary and hence the nucleic acid domains of the proximity probes cannot interact or form a duplex directly. Instead, a third nucleic acid molecule is provided, namely a “splint” oligonucleotide as discussed above. Thus, when the proximity probes bind to their respective analyte, the nucleic acid domains of the probes each interact by hybridisation, i.e. form a duplex, with the splint oligonucleotide.

In accordance with Version 3, it can be seen therefore that the third nucleic acid molecule or splint may be regarded as the second strand of a partially double stranded nucleic domain provided on one of the proximity probes. In this embodiment the nucleic acid domain of the second proximity probe (which has a free 3′ end) may be extended using the “splint oligonucleotide” as a template. Alternatively or additionally, the free 3′ end of the splint oligonucleotide (i.e. the unattached strand, or the 3′ single-stranded region of the first proximity probe) may be extended using the nucleic acid domain of the second proximity probe as a template.

As discussed above in connection with Version 3, the splint oligonucleotide may be provided as a separate component of the assay or the splint may be pre-hybridised to one of the nucleic acid domains of the proximity probes, i.e. hybridised prior to contacting the proximity probe with the sample.

Hence, in this embodiment also, as discussed above, the extension of the nucleic acid domain of the proximity probes as defined herein encompasses also the extension of the “splint” oligonucleotide.

Whilst the splint oligonucleotide depicted in Versions 3 and 5 of FIG. 1 is shown as being complementary to the full length of the nucleic acid domain of the first proximity probe, this is merely an example and it is sufficient for the splint to be capable of forming a duplex with the ends (or near the ends) of the nucleic acid domains of the proximity probes, i.e. to form a bridge between the nucleic acid domains of the proximity probes.

Version 6 of FIG. 1 represents a version of PEA of particular interest. That is to say, when the method is performed within the context of a PEA, or includes a PEA, in a particular representative embodiment the PEA is performed in accordance with Version 6 of FIG. 1. As depicted, in this version both probes in a pair are conjugated to partially single-stranded nucleic acid molecules. In each probe a short nucleic acid strand is conjugated via its 5′ end to the analyte-binding domain (though the strands can be conjugated via their 3′ ends to the analyte-binding domains instead). The short nucleic acid strands which are conjugated to the analyte-binding domains do not hybridise to each other. Rather, each short nucleic acid strand is hybridised to a longer nucleic acid strand, which has a single-stranded overhang at its 3′ end (that is to say, the 3′ end of the longer nucleic acid strand extends beyond the 5′ end of the shorter strand conjugated to the analyte-binding domain. The overhangs of the two longer nucleic acid strands hybridise to one another, forming a duplex. If the 3′ ends of the two longer nucleic acid molecules hybridise fully to one another, as shown, the duplex comprises two free 3′ ends, though the 3′ ends of the longer nucleic acid molecules may be designed as in Version 4, such that the extreme 3′ end of one of the longer nucleic acid molecules is not complementary to the other, forming a flap, meaning that the duplex contains only one free 3′ end. The two longer nucleic acid molecules which interact with one another may be seen as splint oligonucleotides, in that together they form a bridge between the two short oligonucleotides which are directly conjugated to the analyte-binding domains.

Addition or activation of a nucleic acid polymerase results in extension of the free 3′ end or ends of the splint oligonucleotides. Notably, extension of either splint oligonucleotide uses the other splint oligonucleotide as template. Thus, when one splint oligonucleotide is extended, the other “template” splint oligonucleotide is displaced from the shorter strand which is conjugated to the analyte-binding domain.

In a particular embodiment, the short nucleic acid strand conjugated directly to the analyte-binding domain is a “universal strand”. That is to say, the same strand is conjugated directly to every proximity probe used in the multiplex detection assay. Each splint oligonucleotide therefore comprises a “universal site”, which consists of the sequence which hybridises to the universal strand, and a “unique site”, which comprises a barcode sequence unique to the probe. In this embodiment, the universal site is located at the 5′ end of each splint oligonucleotide and the unique site at the 3′ end. Such proximity probes, and methods for making them, are described in WO 2017/068116.

In all proximity detection assay techniques, in certain embodiments the nucleic acid domain of each individual proximity probe comprises a unique barcode sequence, which identifies the particular probe (as described above for PEA Version 6). In this case, the reporter nucleic acid molecule (which in the context of proximity extension assays is the extension product) comprises the unique barcode sequence of each proximity probe. These two unique barcode sequences thus together form the barcode sequence of the reporter nucleic molecule. In other words, the reporter nucleic acid molecule barcode sequence comprises a combination of two probe barcode sequences, from the proximity probes which combined to generate the reporter nucleic acid molecule. Detection of a particular reporter sequence is thus achieved by detecting a particular combination of two probe barcode sequences. In this respect, as noted above the barcode sequence of an individual proximity probe may be seen as a partial barcode sequence of the reporter molecule.

As detailed above, proximity extension assays comprise an extension step performed immediately after the binding of probes to their targets. The extension step forms the initial copies of the reporter nucleic acid molecules generated in the assay. The extension step is performed using a nucleic acid polymerase. Following the extension step an amplification step may be performed, in order to amplify the reporter nucleic acid molecules generated in the extension step. The amplification step is generally performed by PCR.

In an embodiment the PEAs comprise a single PCR, which comprises both the extension step and the amplification step of the PEA. That is to say, the PEA may comprise an extension step that generates the reporter DNA molecules, and an amplification step in which the reporter DNA molecules are amplified, and the extension and amplification steps take place within a single PCR. In this embodiment, rather than beginning with a denaturation step (as is normally the case in PCR), the reaction begins with an extension step, during which the reporter nucleic acid molecule is generated. Thereafter, a standard PCR is performed to amplify the reporter nucleic acid molecule, beginning with denaturation of the reporter molecule. As detailed above, in an embodiment every reporter DNA molecule is generated using proximity probes comprising nucleic acid domains comprising a 5′ universal site and a 3′ unique site. This means that in this embodiment, every reporter DNA molecule has universal end sequences flanking a central barcode sequences. In an embodiment the two universal end sequences are different, i.e. every reporter DNA molecule comprises a first universal end sequence at one end and a second universal end sequence at the other end. The amplification reaction can thus be performed with a single common set of primers that hybridise to the universal end sequences of the reporter DNA molecules, and therefore function to amplify all reporter DNA molecules. The same set of universal (common) primers can be used for the amplification step (i.e. the first PCR) in all pools.

Thus in an embodiment, there is provided a method for detecting multiple analytes in one or more samples, the method comprising:

(i) performing multiple separate multiplex proximity extension assays, wherein each multiplex proximity extension assay detects multiple analytes in a sample, and each multiplex detection assay generates a pool of reporter DNA molecules, each of which is specific for a particular analyte;

wherein each proximity extension assay comprises a first PCR, the first PCR comprising an extension step in which the reporter DNA molecules are generated, and an amplification step in which the reporter DNA molecules are amplified;

(ii) in each pool, performing a second PCR wherein the reporter DNA molecules are modified by the addition of defined end sequences which may be joined in a concatenation step, the reporter DNA molecules in the same pool all having the same end sequences and the different pools having different end sequences, such that a reporter DNA molecule from one pool may only be joined to a reporter DNA molecule from one or two pre-determined different pools;

(iii) combining the pools;

(iv) generating multiple linear DNA concatemers of a pre-defined length, wherein each concatemer is generated by joining together one random reporter DNA molecule from each pool in a pre-determined order such that the position of each reporter DNA molecule within the concatemer indicates the pool from which it is derived and each concatemer comprises a pre-determined number of reporter DNA molecules; and

(v) sequencing the concatemers, thereby detecting a reporter DNA sequence from each pool in each concatemer, wherein the reporter DNA sequence from each pool is assigned to that pool based upon its position within its concatemer, and thereby detecting the analytes in the or each sample.

As noted above, the reporter DNA molecules may be generated with universal (common) end sequences. Each second PCR can therefore be performed with a single pair of universal primers, capable of hybridising to and amplifying all reporter DNA molecules. However, unlike in the first PCR where a single primer pair can be used in all pools, in the second PCR a different primer pair is used in each separate pool, each primer pair comprising the same 3′ hybridisation sites and a different pair of 5′ defined end sequences.

In a particular embodiment, the multiple multiplex PEAs are performed to detect different sets of analytes in the same sample. Thus multiple multiplex PEAs are performed on a single sample, each PEA using a different panel of proximity probe pairs. Each panel of proximity probe pairs comprises a different set of proximity probe pairs. That is to say, the proximity probe pairs in each panel bind a different set of analytes. In general, the proximity probe pairs in each panel bind a completely different set of analytes, i.e. there is no overlap in analytes bound by the proximity probe pairs in different panels. It can thus be seen that each panel of proximity probes is for the detection of a different group of analytes.

As noted above, each panel of proximity probes comprises a different set of proximity probe pairs. Within each individual panel, every probe comprises a different nucleic acid domain (i.e. every probe comprises a nucleic acid domain with a different sequence). Thus every probe pair comprises a different pair of nucleic acid domains, and so a unique reporter DNA molecule is generated for each probe pair within a panel. However, the same nucleic acid domains (and generally the same nucleic acid domain pairings) are used in the probe pairs in each different panel. That is to say, in different panels the probe pairs comprise the same pairs of nucleic acid domains. This means that the same reporter DNA molecules are generated in every panel. However, because the reporter DNA molecules are generated by each panel using different probe pairs, the same reporter DNA molecule denotes the presence of a different analyte in each panel of probes.

Since a different panel of proximity probe pairs is used for each of the multiplex PEAs, each pool of reporter DNA molecules is formed from one panel of proximity probe pairs. Following concatenation, it is therefore known that all reporter DNA sequences denote the presence of a particular analyte in the sample. Upon concatemer sequencing, the position of each reporter DNA sequence within a concatemer provides the information as to which analyte the sequence denotes the presence of within the sample.

This embodiment can therefore be seen to provide a method as described immediately above, in which the multiple multiplex proximity extension assays are performed on the same sample; and

wherein each proximity extension assay comprises detecting analytes using pairs of proximity probes, each proximity probe comprising:

(i) an analyte-binding domain specific for an analyte; and

(ii) a nucleic acid domain,

wherein both probes within each pair comprise analyte-binding domains specific for the same analyte, and each probe pair is specific for a different analyte, and wherein each probe pair is designed such that on proximal binding of the pair of proximity probes to their respective analyte the nucleic acid domains of the proximity probes interact to generate a reporter DNA molecule;

wherein at least 2 panels of proximity probe pairs are used, each panel being for the detection of a different group of analytes, and each multiplex proximity extension assay uses one panel of proximity probe pairs;

wherein (a) within each panel, every probe pair comprises a different pair of nucleic acid domains; and (b) in different panels the probe pairs comprise the same pairs of nucleic acid domains; and

wherein the product of each panel of proximity probe pairs forms a pool.

Reference to the nucleic acid domains of the proximity probes interacting to generate a reporter DNA molecule means that the nucleic acid domains of the proximity probes hybridise to one another, such that they are capable of forming a template or the templates for an extension reaction. A PCR is then performed comprising first an extension step to generate the reporter DNA molecules, followed by an amplification step for amplification of the reporter DNA molecules.

In an alternative embodiment, the multiple multiplex PEAs are performed to detect the same sets of analytes in multiple different samples. In this embodiment, each PEA utilises the same set (i.e. panel) of proximity probe pairs, and each PEA is performed on a different sample. As described above, each PEA generates a pool of reporter DNA molecules, which are subsequently concatenated and sequenced. Since the same panel of proximity probe pairs is used in each PEA, each reporter DNA sequence is known to denote a specific analyte (which is the same across all pools). Thus upon concatemer sequencing, the position of each reporter DNA sequence within a concatemer provides the information as to which sample the denoted analyte is present in.

As also detailed above, in another alternative embodiment the multiple multiplex PEAs are performed to detect multiple sets of analytes in multiple different samples. For example, two sets of analytes could be detected in two different samples, requiring a total of four multiplex PEA reactions. As detailed above, each of the two sets of analytes would be detected using a different panel of proximity probe pairs, and thus two sets of proximity probe pairs would be required for analysis of each of the two samples. In this embodiment, following concatenation and sequencing, the location of each reporter DNA sequence in a concatemer would provide the information as to both the denoted analyte (depending on the panel of proximity probe pairs from which the reporter molecule was generated) and the sample in which the analyte was present.

As detailed above, concatenation can be performed using any suitable method known in the art. In a particular and preferred embodiment, concatenation is performed by USER assembly. The basic principle of USER assembly has been known for several years and is described in Geu-Flores et al., Nucleic Acids Research 35(7): e55, 2007; and an improved protocol was described in Lund et al., PLoS ONE 9(5): e96693, 2014. Both documents are incorporated by reference. USER stands for uracil-specific excision reagent, and is a means of directed assembly of multiple DNA fragments without any requirement for the use of restriction enzymes.

In USER assembly, the DNA fragments to be assembled are provided with double-stranded extensions at their ends (or at least at whichever end(s) is/are to be fused to another DNA fragment in the assembly reaction). The extension sequences comprise unique assembly sites. Each double-stranded extension has a first strand comprising at least one (preferably multiple) uracil residues, while the second strand contains only the standard DNA bases (uracil residues in the first strand being paired with adenine residues in the second strand). In DNA fragments that are to be fused, the assembly site sequences in the strands of the extensions that do not contain uracil residues are complementary. Generally, the extensions are provided to the DNA fragments to be assembled by PCR using primers containing 5′ assembly sites which include the uracil nucleotide(s). In each extension, the uracil residues are therefore generally in the 5′ strand (i.e. the strand with its 5′ end at the end of the extension).

Assembly of DNA fragments is performed by application of the USER enzyme mix (Uracil DNA glycosidase (UDG) and DNA glycosylase-lyase endo VIII (EndoVIII)). UDG cleaves the glycosidic bond within a uracil nucleotide between the uracil base moiety and the deoxyribosy sugar moiety, causing loss of the uracil base from the nucleotide and forming an abasic site. EndoVIII recognizes the abasic site created by UDG and cleaves the phosphodiester bonds 3′ and 5′ of the abasic site to create a nick in the DNA at that location. Excision of the uracil nucleotide by the USER enzyme mix destabilises the double helix of the DNA strand, resulting in loss of the short sequence upstream of the nick from the nicked strand, resulting in a single-stranded 3′ overhang. Heating of the DNA molecules after the uracil excision can enhance destabilisation, improving overhang formation. Similarly, the inclusion of multiple uracil residues in the assembly site results in the formation of multiple nicks in the DNA and enhanced destabilisation.

Following the generation of single-stranded 3′ overhangs, the complementary overhangs of DNA fragments that are to be fused hybridise to one another, and are ligated together (using DNA ligase).

In the method, the assembly sites are added to the DNA molecules (e.g. reporter DNA molecules) by PCR. The PCR is performed using primers which comprise a 3′ hybridisation site (which hybridises to the target DNA molecule), and a 5′ assembly site. Such primers are referred to herein as assembly primers. The 5′ assembly site of the primer provides the defined end sequence. It may be viewed as a “pool-specific” portion of the primer. The 3′ hybridisation site may be viewed as the “universal” portion of the assembly primer. The 5′ assembly sites in the primers each comprise at least one uracil residue, preferably multiple uracil residues. For instance, each assembly site may comprise at least two uracil residues, more preferably at least 3 uracil residues. When an assembly site comprises multiple uracil residues, the uracil residues may be next to one another, or may be spread out across the assembly site, being separated by other, non-uracil residues. One uracil residue must be located at the 3′ end of the assembly site, so that following application of the USER mix the generated 3′ overhang comprises the entire assembly site.

Thus a PCR is performed on each pool of DNA molecules using assembly primers. In line with the teaching above, the assembly primers used in each pool comprise at most a single pair of assembly sites, i.e. in each pool the forward primer (or primers) comprises (or comprise) a first assembly site and the reverse primer (or primers) comprises (or comprise) a second, different assembly site. In particular all the DNA molecules within each pool comprise a pair of common primer binding sites, such that a single pair of assembly primers can be used to amplify all the DNA molecules in each pool. The PCRs performed on the pools of DNA molecules that are intended to form the ends of the concatemers may be performed using a primer pair comprising one assembly primer and one standard primer (i.e. not comprising an assembly site), depending on whether an additional assembly site is desired at the end of the concatemer. In particular, all pools of DNA molecules are subjected to PCRs utilising a pair of assembly primers.

In line with the teaching above, different assembly sites are provided in the primers used for the PCR performed in each different pool. However, complementary assembly sites are provided to the DNA molecules in pools which are intended to be joined to one another, such that when the pools are combined the DNA molecules intended to join to one another hybridise to each other via their assembly sites, and are then ligated together, thus forming concatemers.

During PCR using assembly primers, amplification of the assembly sites proceeds using standard DNA nucleotides, with adenine residues paired with the uracil residues from the assembly primers. The PCR thus generates DNA products comprising assembly sites at both ends (except, potentially, in the case of DNA molecules intended to form the ends of the concatemers, which as noted above may only have an (end sequence) assembly site at one end), wherein the assembly site at the 5′ end of each strand (which originates from an assembly primer) comprises at least one uracil residue, while the complementary assembly sites at the 3′ ends of the strands comprise only the standard DNA bases. Treatment of the resulting DNA products with the USER enzyme mix thus results in DNA products having a 3′ overhang on each strand, which can then hybridise to complementary 3′ overhangs in the DNA molecules of other pools.

In an alternative embodiment, concatenation is performed by Gibson assembly. Gibson assembly is described in Gibson et al., Nature Methods 6: 343-345, 2009; and Gibson et al., Science 329: 52-56, 2010, both incorporated herein by reference. Similarly to USER assembly, Gibson assembly of DNA fragments is performed by generating DNA fragments with overlapping ends. Commonly the fragments are generated by performing PCR using assembly primers comprising 5′ assembly sites that form the overlapping ends of DNA fragments that are to be joined. The DNA fragments are mixed together and the Gibson enzyme mix applied, which contains DNA exonuclease, DNA polymerase and DNA ligase. The exonuclease degrades DNA from the 5′ ends of each fragment, resulting in 3′ overhangs at the ends of each fragment. The overhangs hybridise to one another, and any gaps between DNA strands following hybridisation are filled in by the DNA polymerase. The strands are then joined by the DNA ligase.

Thus while the Gibson and USER assembly techniques have differences, both utilise assembly sites at the termini of the DNA molecules to be assembled, which are generally introduced into the DNA molecules by PCR using assembly primers. In both cases, 3′ overhangs are generated at the ends of DNA molecules, which hybridise to complementary 3′ overhangs in other DNA molecules which are to be joined to them.

Thus in a particular embodiment, the method comprises performing a PCR on each pool using assembly primers, wherein all the DNA molecules in each pool are amplified using the same primer pair, and a different primer pair is used for amplification in each pool, and each species of assembly primer comprises a unique assembly site (or “pool-specific” portion), such that all the PCR products in each pool comprise a unique pre-defined assembly site at one or both ends; and

wherein in the concatenation step, the PCR products of each pool are joined to the PCR products of different pools having complementary assembly sites, thereby generating the concatemers.

That is to say, provided herein is a method of detecting DNA sequences from multiple pools, wherein each pool comprises multiple species of DNA molecule, the method comprising:

(i) performing a PCR on each pool using an assembly primer pair, wherein all the DNA molecules in each pool are amplified using the same primer pair, and a different primer pair is used for amplification in each pool, and each species of assembly primer comprises a unique assembly site, such that all the PCR products in each pool comprise a unique pre-defined assembly site at one or both ends;

and wherein the assembly sites are suitable for joining of the PCR products by USER assembly or Gibson assembly;

(ii) combining the pools;

(iii) generating multiple linear DNA concatemers of a pre-defined length, wherein each concatemer is generated by joining together one random DNA molecule from each pool in a pre-determined order, the PCR products of each pool being joined to the PCR products of different pools having complementary assembly sites, such that the position of each DNA molecule within the concatemer indicates the pool from which it is derived and each concatemer comprises a pre-determined number of DNA molecules;

wherein the concatemers are generated by USER assembly or Gibson assembly; and

(iv) sequencing the concatemers, thereby detecting a DNA sequence from each pool in each concatemer, wherein the DNA sequence from each pool is assigned to that pool based upon its position within its concatemer.

As noted above, in this embodiment all the DNA molecules in each pool are amplified using the same primer pair. That is to say, the PCR reaction in each pool utilises one forward primer and one reverse primer. This means that all DNA molecules in each pool comprise common primer binding sites, such that all DNA molecules in each pool can be amplified using a single set of primers. In a particular embodiment, all DNA molecules across all pools comprise the same common primer binding sites, such that all primers used in the method comprise the same hybridisation sites (or “universal” portions) and differ only by their assembly sites.

An assembly primer pair comprises at least one assembly primer. As detailed above, an assembly primer comprises a 3′ hybridisation site (“universal” site) and a 5′ assembly site (“pool-specific” portion). In some or all assembly primer pairs both primers are assembly primers, i.e. both primers in a pair may comprise a 5′ assembly site. However, as detailed above, in the assembly primer pairs used to amplify the DNA molecules in the pools which are to form the ends of the concatemers, only one of the two primers in the assembly primer pair must be an assembly primer (i.e. must comprise an assembly site), depending on whether an assembly site is desired at the relevant end of the concatemer. However, in a particular embodiment all assembly primer pairs comprise two assembly primers, i.e. that both primers in the pair comprise assembly sites. This results in assembly sites being present at the ends of the concatemers formed, for further assembly to take place.

Since all the DNA molecules in each pool are amplified using the same primer pair, all the PCR products generated in each pool comprise the same assembly site(s).

As detailed, a different primer pair is used for amplification in each pool. By “different” in this respect means that no specific primer is used in two or more different pools. Every primer used across all amplification reactions is used in only one pool, such that the two primers used for amplification in any given pool are unique and different to any primer (i.e. have a different sequence to any primer) used for amplification in any of the other pools.

A “species of primer” as used herein refers to a primer of a particular sequence (and thus a “species of assembly primer” refers to an assembly primer of a particular sequence). Each PCR thus utilises two species of primer, and as noted above the two species of primer used in each PCR are unique, each species of primer being used only in a single PCR performed on one pool. As noted above, in particular embodiments the primer hybridisation sequences are shared across all pools, such that all species of primers of a given orientation (i.e. “forward” or “reverse”) used across all the pools have the same hybridisation site. However, as noted above every species of assembly primer comprises a unique assembly site. An “assembly site” as used herein is defined as a sequence that is used for a particular DNA molecule (from a particular pool) to hybridise to another DNA molecule (from a pre-defined other pool). Where the assembly site is introduced into the DNA molecules by PCR, as in the present embodiment, the assembly site is located at the 5′ end of a primer and does not overlap with the hybridisation site. In particular, where the DNA molecules are reporter DNA molecules generated in a detection assay, the assembly sites are not present in the reporter DNA molecules when they are first generated, but are only introduced in a PCR step. In particular, the assembly sites do not form part of the reporter DNA molecule barcode sequences. Since the assembly sites are located at the 5′ ends of the assembly primers used to introduce the sites, in the resulting PCR products the assembly sites are located at the termini.

Each species of assembly primer used across the pools comprises a unique assembly site. That is to say, each species of assembly primer comprises an assembly site with a unique sequence, such that no two species of assembly primer comprise the same assembly site sequence. This is, of course, essential in order for DNA molecules from each pool to be located at a defined position within the concatemers. However, while no two species of assembly primer comprise the same assembly site sequence, as discussed above, complementary pairs of assembly sites are used across the pools. PCR products comprising complementary assembly sites are thus able to hybridise to one another and be joined. Thus every assembly site used within the PCRs across the pools has a paired, complementary assembly site. Pairs of complementary assembly sites are used in PCRs on different pools, i.e. a single PCR performed on a particular pool never uses primers with complementary assembly sites. This could result in circularisation of the PCR products, which would not then be suitable for concatenation.

Thus as explained above, each PCR is performed with a different assembly primer pair, such that the resulting PCR products each contain a unique pre-defined assembly site at one or both ends. By “pre-defined” is meant that the assembly site to be added to a particular end of the DNA molecules in a given pool is selected and thus known in advance of the PCR being performed. Because unique pre-defined assembly sites are added to the DNA molecules in each pool, complementary assembly sites can be intentionally added to the ends of DNA molecules in different pool such that they will hybridise and be joined to one another. The order in which DNA molecules from the different pools will be joined during the concatenation reaction is thus pre-defined, based on the arrangement of complementary assembly sites across the pools. The PCR products of each pool are thus joined to the PCR products of pre-defined different pools during the concatenation step, determined by which different pools comprise PCR products having complementary assembly sites.

As noted above, concatenation may in particular be performed by USER assembly. When USER assembly is used for concatenation, in particular each assembly site across all species of assembly primers comprises multiple uracil residues, and more particularly all assembly sites comprise at least 3 uracil residues.

As detailed above, once the PCRs have been performed to introduce the assembly sites into the DNA molecules in each pool, the PCR products are processed with an enzyme (or enzyme mixture) to generate 3′ overhangs required for concatenation. When USER assembly is used for concatenation, the 3′ overhangs are generated using the USER enzyme mix (UDG and EndoVIII), whereas when Gibson assembly is used the 3′ overhangs are generated with an exonuclease. This step of generating the 3′ overhangs can be performed before or after the pools are combined.

In an embodiment, the 3′ overhangs are generated before the pools are combined. In this embodiment, a PCR is performed on each pool using assembly primers. Following the PCR, the products are treated with the appropriate enzyme or enzyme mix (depending on the method used for concatenation) in order to generate 3′ overhangs. The pools are then combined so that DNA molecules from the various pools are able to hybridise to each other via their complementary 3′ overhangs. The hybridised DNA molecules are then joined to each other in order to form concatemers, the joining is performed using the appropriate enzyme or enzyme mix (depending on the method used for concatenation): when USER assembly is used for concatenation, the hybridised DNA molecules are joined by DNA ligase alone; when Gibson assembly is used for concatenation, the hybridised DNA molecules are joined by a combination of DNA polymerase (to fill in any gaps between strands) and DNA ligase.

Thus in this embodiment, there is provided a method of detecting DNA sequences from multiple pools, wherein each pool comprises multiple species of DNA molecule, the method comprising:

(i) performing a PCR on each pool using an assembly primer pair, wherein all the DNA molecules in each pool are amplified using the same primer pair, and a different primer pair is used for amplification in each pool, and each species of assembly primer comprises a unique assembly site, such that all the PCR products in each pool comprise a unique pre-defined assembly site at one or both ends;

and wherein the assembly sites are suitable for joining of the PCR products by USER assembly or Gibson assembly;

(ii) assembling the PCR products from the pools into linear concatemers by USER assembly or Gibson assembly, the assembly step comprising:
(a) processing the PCR products in each pool to generate 3′ overhangs comprising the assembly sites;
(b) combining the pools; and
(c) generating multiple linear DNA concatemers of a pre-defined length, wherein each concatemer is generated by joining together one random DNA molecule from each pool in a pre-determined order, the PCR products of each pool being joined to the PCR products of different pools having complementary 3′ overhangs, such that the position of each DNA molecule within the concatemer indicates the pool from which it is derived and each concatemer comprises a pre-determined number of DNA molecules;

(iii) sequencing the concatemers, thereby detecting a DNA sequence from each pool in each concatemer, wherein the DNA sequence from each pool is assigned to that pool based upon its position within its concatemer.

Alternatively, as described above the 3′ overhangs in the PCR products can be generated following the combination of the PCR products. In this case, all the necessary assembly enzymes (i.e. the USER mix plus DNA ligase, or the Gibson mix) can be added to together to the combined PCR products.

As described above, in particular embodiments the DNA molecules to be joined are reporter DNA molecules generated in PEAs performed to detect analytes in one or more samples. Thus in a particular embodiment, provided herein is a method for detecting multiple analytes in one or more samples, the method comprising:

(i) performing multiple multiplex proximity extension assays, thereby generating multiple pools of reporter DNA molecules, wherein the reporter DNA molecules in each pool comprise universal primer binding sites at their 3′ and 5′ termini;

(ii) performing a PCR on each pool using an assembly primer pair, wherein all the DNA molecules in each pool are amplified using the same primer pair, and a different primer pair is used for amplification in each pool, and each species of assembly primer comprises a unique assembly site, such that all the PCR products in each pool comprise a unique pre-defined assembly site at one or both ends;

wherein the assembly sites are suitable for USER assembly such that the PCR products from each pool can be joined to the PCR products from one or two different pools;

(iii) assembling the PCR products from the pools into linear concatemers by USER assembly, the assembly step comprising:
(a) processing the PCR products in each pool to generate 3′ overhangs comprising the assembly sites;
(b) combining the pools; and
(c) generating multiple linear DNA concatemers of a pre-defined length, wherein each concatemer is generated by joining together one random DNA molecule from each pool in a pre-determined order, the PCR products of each pool being joined to the PCR products of different pools having complementary 3′ overhangs, such that the position of each DNA molecule within the concatemer indicates the pool from which it is derived and each concatemer comprises a pre-determined number of DNA molecules;

(iv) sequencing the concatemers, thereby detecting a DNA sequence from each pool in each concatemer, wherein the DNA sequence from each pool is assigned to that pool based upon its position within its concatemer, and thereby detecting the analytes in the or each sample.

More generally, provided herein is a method for detecting multiple analytes in one or more samples, the method comprising:

(i) performing multiple multiplex proximity extension assays, thereby generating multiple pools of reporter DNA molecules, wherein the reporter DNA molecules in each pool comprise universal primer binding sites at their 3′ and 5′ termini;

(ii) performing a PCR on each pool using assembly primers comprising assembly sites for USER assembly;

(iii) combining the PCR products of each pool and generating multiple linear DNA concatemers of a pre-defined length by USER assembly, wherein each concatemer is generated by joining together one random DNA molecule from each pool in a pre-determined order, such that the position of each DNA molecule within the concatemer indicates the pool from which it is derived and each concatemer comprises a pre-determined number of DNA molecules; and

(iv) sequencing the concatemers, thereby detecting a DNA sequence from each pool in each concatemer, wherein the DNA sequence from each pool is assigned to that pool based upon its position within its concatemer, and thereby detecting the analytes in the or each sample.

As detailed above, after being generated the concatemers are sequenced. Conveniently, a form of high throughput DNA sequencing may be used in this step. Sequencing by synthesis is an example of a DNA sequencing method that may be used in the method provided herein. Examples of sequencing by synthesis techniques include pyrosequencing, reversible dye terminator sequencing and ion torrent sequencing, any of which may be utilised in the present method. In an embodiment, the concatemers are sequenced using massively parallel DNA sequencing. Massively parallel DNA sequencing may in particular be applied to sequencing by synthesis (e.g. reversible dye terminator sequencing, pyrosequencing or ion torrent sequencing, as mentioned above). Massively parallel DNA sequencing using the reversible dye terminator method is a convenient sequencing method for use in the method provided herein. Massively parallel DNA sequencing using the reversible dye terminator method may be performed, for instance, using an Illumina® NovaSeq™ system.

As is known in the art, massively parallel DNA sequencing is a technique in which multiple (e.g. thousands or millions or more) DNA strands are sequenced in parallel, i.e. at the same time. Massively parallel DNA sequencing requires target DNA molecules to be immobilised to a solid surface, e.g. to the surface of a flow cell or to a bead. Each immobilised DNA molecule is then individually sequenced. Generally, massively parallel DNA sequencing employing reversible dye terminator sequencing utilises a flow cell as the immobilisation surface, and massively parallel DNA sequencing employing pyrosequencing or ion torrent sequencing utilises a bead as the immobilisation surface.

As is known to the skilled person, immobilisation of DNA molecules to a surface in the context of massively parallel sequencing is generally achieved by the attachment of one or more sequencing adapters to the ends of the molecules. The method may thus include the addition of one or more adapters for sequencing (sequencing adapters) to the concatemers.

Commonly, sequencing adapters are nucleic acid molecules (in particular DNA molecules). In this instance, short oligonucleotides complementary to the adapter sequences are conjugated to the immobilisation surface (e.g. the surface of the bead or flow cell) to enable annealing of the target DNA molecules to the surface, via the adapter sequences. Alternatively, any other pair of binding partners may be used to conjugate the target DNA molecule to the immobilisation surface, e.g. biotin and avidin/streptavidin. In this case biotin may be used as the sequencing adapter, and avidin or streptavidin conjugated to the immobilisation surface to bind the biotin sequencing adapter, or vice versa.

Sequencing adapters may thus be short oligonucleotides (preferably DNA), generally 10-30 nucleotides long (e.g. 15-25 or 20-25 nucleotides long). As detailed above, the purpose of a sequencing adapter is to enable annealing of the target DNA molecules to an immobilisation surface, and accordingly the nucleotide sequence of a nucleic acid sequencing adaptor is determined by the sequence of its binding partner conjugated to the immobilisation surface. Aside from this, there is no particular constraint on the nucleotide sequence of a nucleic acid sequencing adaptor.

A sequencing adapter may be added to a concatemer during PCR amplification, as detailed further below. In the case of a nucleic acid sequencing adapter this can be achieved by including a sequencing adapter nucleotide within in one or both primers. Alternatively, if the sequencing adaptor is a non-nucleic acid sequencing adaptor (e.g. a protein/peptide or small molecule) an adapter may be conjugated to one or both PCR primers. Alternatively, a sequencing adapter may be attached to a concatemer by directly ligating or conjugating the sequencing adapter to the concatemer. In a particular embodiment sequencing adapters are added to both ends of the concatemers during the concatenation process. That is to say, an assembly site may be added to each of the sequencing adapters, as described above, combined with the pools of DNA molecules, and assembled into concatemers as described above (such that the sequencing adapters form the ends of the concatemers). Particularly, the one or more sequencing adapters used in the present method are nucleic acid sequencing adapters, specifically DNA sequencing adaptors.

Thus one or more nucleic acid sequencing adapters may be added to the concatemers in an amplification step. In particular, the concatemers may be subjected to a PCR to add at least a first sequencing adapter to the concatemers. Preferably, two sequencing adapters are added to the concatemers (one at each end) within a single PCR (i.e. by PCR amplification using a pair of primers which both contain a sequencing adapter), though two amplification steps may alternatively be performed (such that a first PCR is performed to add a first sequencing adapter to the concatemers, followed by a second PCR to add a second sequencing adapter to the other end of the concatemers). Generally, when two sequencing adapters are added to the concatemers, different sequencing adapters are added at each end.

As noted above, one or more sequencing adapters may be added to the concatemers. By this is meant one or two sequencing adapters—since sequencing adapters are added to the ends of a DNA molecule, the maximum number of sequencing adapters which can be added to a single DNA molecule (in this instance, concatemer) is two. Thus a single sequencing adapter may be added to one end of a concatemer, or two sequencing adapters may be added to a concatemer, one to each end. In a particular embodiment the IIlumina P5 and P7 adapters are used, i.e. the P5 adapter is added to one end of the concatemer and the P7 adapter is added to the other end. The sequence of the P5 adapter is set forth in SEQ ID NO: 1 and the sequence of the P7 adapter is set forth in SEQ ID NO: 2.

In a particular embodiment, following concatemer generation a single PCR is performed to amplify the concatemers and attach sequencing adapters to their ends (i.e. to add a sequencing adapter to both ends of the concatemers). In this embodiment, the PCR is performed using a pair of primers each of which comprises a 5′ sequencing adaptor upstream of the 3′ hybridisation site. See, for example, FIG. 7, showing PCR3.

When sequencing adapters are added to the ends of the concatemers, the sequencing adapters are used in the sequencing step to immobilise the concatemers onto a surface for sequencing.

As detailed above, in an embodiment the concatemers are assembled from DNA molecules that have assembly sites at both ends, such that the resulting concatemer has assembly sites at both ends. In an embodiment the primers used for the PCR performed to attach sequencing adaptors to the concatemers hybridise to the terminal assembly sites. That is to say, the hybridisation sites of the primers used to add sequencing adaptors to the concatemers may be complementary to the concatemers' terminal assembly sites. As all concatemers contain the same terminal assembly sites, a single primer pair is capable of amplifying all concatemers.

In another embodiment, the concatemers are subjected to a PCR to add at least a first sequencing primer binding site to the concatemers. As is well known in the art, most DNA sequencing techniques, including all those presently used for massively parallel DNA sequencing, utilise a sequencing primer to initiate synthesis of the sequencing strand. A sequencing primer binding site is accordingly a DNA sequence which is complementary to the sequence of a sequencing primer, such that a sequencing primer is capable of hybridising to it. There is no particular constraint on the sequence of the sequencing primer binding site.

Thus one or more sequencing primer binding sites may be added to the concatemers in an amplification step. In particular, the concatemers may be subjected to a PCR to add at least a first sequencing primer binding site to the concatemers. Preferably, two sequencing primer binding sites are added to the concatemers (one at each end) within a single PCR (i.e. by PCR amplification using a pair of primers which both contain a sequencing primer binding site), though two amplification steps may alternatively be performed (such that a first PCR is performed to add a first sequencing primer binding site to the concatemers, followed by a second PCR to add a second sequencing primer binding site to the other end of the concatemers). When two sequencing primer sites are added to the concatemers, generally different sequencing primer binding sites are added at each end, though this is not essential as the same sequencing primer can be used for sequencing of the DNA molecules in both directions. However, the use of different sequencing primer binding sites at each end of the concatemers is preferred, since each strand would otherwise comprise reverse complementary sequencing primer binding sites at its ends, increasing the risk of hairpin structures forming within the concatemer strands.

Rather than using PCR (or other amplification technique) the sequencing primer binding sites may alternatively be assembled into the concatemers during concatenation, as detailed for the sequencing adapters above.

In an embodiment, following concatemer generation a single PCR is performed to amplify the concatemers and attach sequencing primer binding sites to their ends (i.e. to add a sequencing primer binding site to both ends of the concatemers). In this embodiment, the PCR is performed using a pair of primers each of which comprises a 5′ sequencing primer binding site upstream of the 3′ hybridisation site. In a particular embodiment the Read 1 sequencing primer (Rd1SP) and Read 2 sequencing primer (Rd2SP) are used for concatemer sequencing, as demonstrated in the Examples below, i.e. the Rd1SP binding site is added to one end of the concatemer and the Rd2SP binding site is added to the other end. The sequence of the Rd1SP binding site is set forth in SEQ ID NO: 3 and the sequence of the Rd2SP binding site is set forth in SEQ ID NO: 4.

As detailed above, the concatemers may be assembled from DNA molecules that have assembly sites at both ends, such that the resulting concatemer has assembly sites at both ends. In an embodiment, the primers used for the PCR performed to attach sequencing primer binding sites to the concatemers hybridise to the terminal assembly sites. That is to say, the hybridisation sites of the primers used to add sequencing primer binding sites to the concatemers may be complementary to the concatemers' terminal assembly sites.

In a particular embodiment both sequencing adaptors and sequencing primer binding sites are attached to the ends of the concatemers. For example, one sequencing adaptor and one sequencing primer binding site are added to each end of the concatemers. In particular, the sequencing adaptors are added such that they form the termini of the concatemers, with the sequencing primer binding sites immediately downstream of the sequencing adaptors and the DNA molecules of interest which formed the concatemers downstream of the sequencing primer binding sites. As described above, generally the sequencing adaptors and sequencing primer binding sites are added to the concatemers by PCR. Although multiple PCRs may be carried out in order to attach the sequencing adapters and sequencing primer binding sites, in an embodiment a single PCR is performed in order to attach both the sequencing adapters and sequencing primer binding sites to the concatemers. The PCR is then thus performed using primers comprising, from 5′ to 3′ a sequencing adapter, a sequencing primer binding site and a hybridisation site.

Thus in a particular embodiment, there is provided a method of detecting DNA sequences from multiple pools, wherein each pool comprises multiple species of DNA molecule, the method comprising:

(i) performing a PCR on each pool using an assembly primer pair, wherein all the DNA molecules in each pool are amplified using the same primer pair, and a different primer pair is used for amplification in each pool, and each species of assembly primer comprises a unique assembly site, such that all the PCR products in each pool comprise a unique pre-defined assembly site at one or both ends;

and wherein the assembly sites are suitable for joining of the PCR products by USER assembly;

(ii) combining the PCR products of each pool and generating multiple linear DNA concatemers of a pre-defined length by USER assembly, wherein each concatemer is generated by joining together one random DNA molecule from each pool in a pre-determined order, such that the position of each DNA molecule within the concatemer indicates the pool from which it is derived and each concatemer comprises a pre-determined number of DNA molecules;

(iii) subjecting the concatemers to a PCR to add a sequencing adapter and a sequencing primer binding site to each end of the concatemers, the PCR being performed with a pair of primers each of which comprises, from 5′ to 3′ a sequencing adapter, a sequencing primer binding site and a hybridisation site; and

(iv) sequencing the concatemers by massively parallel DNA sequencing, thereby detecting a DNA sequence from each pool in each concatemer, wherein the DNA sequence from each pool is assigned to that pool based upon its position within its concatemer.

In another embodiment, there is provided a method for detecting multiple analytes in one or more samples, the method comprising:

(i) performing multiple multiplex proximity extension assays, thereby generating multiple pools of reporter DNA molecules, wherein the reporter DNA molecules in each pool comprise universal primer binding sites at their 3′ and 5′ termini;

(ii) performing a PCR on each pool using an assembly primer pair, wherein all the DNA molecules in each pool are amplified using the same primer pair, and a different primer pair is used for amplification in each pool, and each species of assembly primer comprises a unique assembly site, such that all the PCR products in each pool comprise a unique pre-defined assembly site at one or both ends;

wherein the assembly sites are suitable for USER assembly such that the PCR products from each pool can be joined to the PCR products from one or two different pools;

(iii) combining the PCR products of each pool and generating multiple linear DNA concatemers of a pre-defined length by USER assembly, wherein each concatemer is generated by joining together one random DNA molecule from each pool in a pre-determined order, such that the position of each DNA molecule within the concatemer indicates the pool from which it is derived and each concatemer comprises a pre-determined number of DNA molecules;

(iv) subjecting the concatemers to a PCR to add a sequencing adapter and a sequencing primer binding site to each end of the concatemers, the PCR being performed with a pair of primers each of which comprises, from 5′ to 3′ a sequencing adapter, a sequencing primer binding site and a hybridisation site; and

(v) sequencing the concatemers by massively parallel DNA sequencing, thereby detecting a DNA sequence from each pool in each concatemer, wherein the DNA sequence from each pool is assigned to that pool based upon its position within its concatemer, and thereby detecting the analytes in the or each sample.

The step of combining the PCR products of each pool and generating multiple linear DNA concatemers of a pre-defined length by USER assembly may be performed as described in more detail above.

In a particular embodiment the method is performed on multiple sets of pools of DNA molecules. The sets of pools may have any relationship. For instance, each set of pools may be derived from a particular sample, with each pool within each sample having been generated by a detection assay to detect a different panel of analytes.

Regardless, in this embodiment, each pool is processed as described above, and the multiple sets of pools are individually combined and a separate concatenation reaction performed for each set of pools, yielding multiple concatenation reaction products. That is to say all the pools from each set are combined, thus forming a separate combined pool from each original set of pools. A separate concatenation reaction is performed for each set of pools, thus generating multiple concatenation reaction products. A concatenation reaction product is the product of a single concatenation reaction.

For increased efficiency it may be desirable to sequence all the concatemers generated in each of the concatenation reactions together. To enable this, a unique index sequence is added to each concatenation reaction product by PCR. Alternatively, the unique index sequences may be incorporated into the concatemers during the concatenation reaction, as described above (i.e. assembly sites may be added to the index sequences, and the sequences combined with the pools of DNA molecules for concatenation). By “unique index sequence” is meant that the same index sequence is added to all the concatemers generated in a particular concatenation reaction (i.e. generated from a particular set of pools) while a different (unique) index sequence is used for each different concatenation reaction product (i.e. for the concatemers generated from each different set of pools), such that the set of pools from which each concatemer originates can be determined by the index sequence contained within the concatemer. The index sequences thus serve to label the concatemers as to the set of pools from which each concatemer originates. The index sequences may be of any length and sequence but are preferably relatively short, e.g. 3-12, 4-10 or 4-8 nucleotides.

Once all concatenation reaction products have been labelled with index sequences, the various concatenation reaction products are combined and sequenced. The sequencing reaction thus identifies the set of pools from which each concatemer originates based on the index sequence contained within the concatemer while the DNA molecules present in the pools within each set can be assigned to their particular pools based on their positions within the concatemers, as detailed above.

As detailed above, the index sequences are added to the concatemers by PCR. Thus a separate PCR reaction is performed for each concatenation reaction in order to add an index sequence to the concatemers. Particularly, two index sequences may be added to each concatemer, one to each end. In this embodiment the PCR is performed with a pair of primers each of which contains an index sequence, i.e. each primer contains a 5′ index sequence and a 3′ hybridisation site. Particularly, the index sequences added to each end of the concatemers are different, e.g. to each concatemer a first index sequence is added to one end and a second index sequence is added to the other end, though the same index sequence can be added to both ends of the concatemers.

In this embodiment, in addition to the index sequence(s), sequencing adaptors and sequencing primer binding sites may be added to the concatemers as discussed above. These elements may be added to the concatemers in separate rounds of PCR. For instance, in one embodiment, the index sequences are added to each of the concatenation reaction products in separate PCRs performed on each concatenation reaction product, the indexed products are then pooled and one or more further PCRs is performed on the pooled, indexed products to add sequencing adapters and sequencing primer binding sites to the concatemers. Alternatively, multiple consecutive PCRs may be separately performed on each concatenation reaction product to sequentially add the index sequences, sequencing primer binding sites and sequencing adaptors. When these three elements are added sequentially, the sequencing adaptors are added last, since the adaptor sequences must be located at the termini of the resulting products, but the index sequences and sequencing primer binding sites may be added in either order.

In an embodiment the three elements (i.e. the index sequences, sequencing primer binding sites and sequencing adaptors) are all added to the concatenation reaction products at the same time, in a single PCR reaction. That is to say, each concatenation reaction product is subjected to a separate PCR in which a sequencing adaptor, sequencing primer binding site and index sequence are added to both ends of the concatemers. This is achieved by performing the PCRs with primer pairs in which each primer comprises a sequencing adaptor, sequencing primer binding site and index sequence upstream of the hybridisation site. In this embodiment, following the PCR the multiple PCR products (which comprise concatemers with a sequencing adaptor, sequencing primer binding site and index sequence at each end) are combined and sequenced.

As described above, in an embodiment, the concatemers are assembled from DNA molecules that have assembly sites at both ends, such that the resulting concatemer has assembly sites at both ends. Conveniently, the primers used for this PCR (i.e. the PCR performed to attach sequencing adaptors, sequencing primer binding sites and index sequences to the concatemers) may hybridise to the terminal assembly sites. That is to say, the hybridisation sites of the primers used in this PCR may be complementary to the concatemers' terminal assembly sites.

As described above, it is required that the sequencing adaptors are added to the concatemers such that they form the termini of the final product that is sequenced. However, the sequencing primer binding sites and index sequences can be arranged in either order. That is to say, the PCR may generate products comprising, at each end, from 5′ to 3′, a sequencing adaptor, a sequencing primer binding site and an index sequence. Alternatively, the PCR may generate products comprising, at each end, from 5′ to 3′, a sequencing adaptor, an index sequence and a sequencing primer binding site. Generally, positioning the index sequence upstream of the sequencing primer binding site may be advantageous when sequencing targets of unknown length (e.g. in genomic sequencing). In this case, the index sequences are read in a specific “index sequencing” reaction that is separate to the main sequencing reaction. However, when the sequencing target is of known length (as in the present method) it is generally advantageous that the index sequence is positioned downstream of the sequencing primer binding site, such that the index sequence can be read at the same time as the sequencing target, such that only a single sequencing reaction needs to be performed to obtain all necessary sequence information from each strand. Accordingly, in an embodiment the PCR to which the concatemers are subjected is designed to yield products comprising, at each end, a sequencing adaptor, a sequencing primer binding site and an index sequence (i.e. products with the index sequence downstream of the sequencing primer binding site). The concatemer of DNA molecules of interest is located downstream of the index sequence. The PCR is thus performed using a primer pair in which each primer comprises, from 5′ to 3′, a sequencing adaptor, a sequencing primer binding site, an index sequence and a hybridisation site.

As detailed above, specific embodiments of the present method comprises several steps. Commonly, the method begins with multiple proximity extension assays. The products of the PEAs are then subjected to PCRs and concatenation reactions (e.g. USER or Gibson assembly), prior to sequencing. The various reactions performed prior to sequencing utilise a number of different enzymes (e.g. DNA polymerase, DNA ligase, UDG, EndoVIII, exonuclease). Enzymatic reactions are generally performed in a buffer that is optimal for activity of the enzyme in question. To perform the method of the invention using, at each stage, a buffer that is optimised for the specific enzyme used in the stage, would however be inefficient. Moreover, the replacement of the buffer at each stage, e.g. by PCR clean-up, would result in substantial loss of product when aggregated through the method. Advantageously, therefore, in an embodiment, all steps prior to sequencing are performed in the same buffer, such that no reaction clean-ups or buffer exchanges are required. Rather, the additional enzyme(s) and/or reagents required at each stage are simply added to the solution sequentially.

Any suitable buffer may be used for this purpose. It is not required that the buffer used is optimised for use with any of the enzymes used in the process, let alone all of them, though it may be the case that all enzymes used in the process have moderate to high activity in the buffer used. The buffer used throughout the process may in particular be a Tris-based buffer.

As noted above, the same buffer may be used in all steps prior to sequencing. If possible, the sequencing reaction may also be performed in the same buffer (such that the entire method utilises only a single buffer). More generally, however, a different buffer is required for the sequencing reaction than is used for the previous method steps. Thus generally prior to sequencing (i.e. after concatenation, or where subsequent PCR steps are performed, after the PCRs to modify the concatemers) the reaction mixture is cleaned up. In other words, the molecules to be sequenced (the concatemers or modified concatemers) are purified and the other parts of the mixtures (buffer, enzymes, nucleotides, etc.) are removed. This can be achieved by any standard method in the art, e.g. using a PCR purification kit, as is available from e.g. Qiagen (Germany). The molecules to be sequenced are then added to a sequencing reaction mix containing the necessary reagents for sequencing, including a specialised sequencing buffer, enzyme etc. Sequencing reagents are commercially available, e.g. from Illumina (USA).

As detailed above, the method of the invention may be used in the context of an analyte detection assay, particularly a PEA. Such detection methods face a challenge when, as is common, the analytes (e.g. proteins of interest) in a sample are present in a wide concentration range, since the signal from analytes of high concentration may overwhelm the signal from analytes of low concentration, resulting in a failure to detect analytes present at lower concentrations. This issue is addressed in co-pending application PCT/EP2021/058008, and the same methods used in that application may be utilised in conjunction with the present method.

Thus in a particular embodiment, the method is used to detect reporter DNA molecules generated in multiple multiplex detection assays (as described above), and the detection assays are performed to detect multiple analytes in one or more samples in which the multiple analytes have a range of levels of abundance. In this embodiment, the detection assay comprises:

(i) providing multiple aliquots from the or each sample; and

(ii) in each aliquot, detecting a different subset of the analytes by performing a separate multiplex assay for each aliquot, wherein the analytes in each subset are selected based on their predicted abundance in the sample.

In particular, in this embodiment the method comprises:

(i) providing multiple aliquots from the or each sample;

(ii) in each aliquot, detecting a different subset of the analytes by performing a separate multiplex detection assay for each aliquot, and generating a first PCR product from each aliquot, wherein the analytes in each subset are selected based on their predicted abundance in the sample;

(iii) combining the first PCR products into multiple pools; and

(iv) performing a second PCR on each pool to modify the first PCR products, to prepare the first PCR products for concatenation.

In this embodiment, the first and second PCRs are as described above. Thus each multiplex detection assay generates reporter DNA molecules, specific for particular analytes, and the first PCR is performed to amplify the reporter DNA molecules generated. The first PCR product is therefore the reporter DNA molecules. The reporter DNA molecules are then combined into multiple pools. The number of pools and the combinations of first PCR products made is dependent on the intended nature of the pools, as discussed above. For instance, if each pool represents a different sample, all the first PCR products (i.e. aliquots) from each sample are combined, thereby yielding a pool for each sample. Alternatively, if each pool represents a different panel of analytes from the same sample (i.e. if each pool represents a detection assay performed with a different panel of proximity probe pairs), all the first PCR products (i.e. aliquots) from each panel are combined, thereby yielding a pool for each panel. In a further alternative, if the method is being used to analyse multiple panels of analytes from multiple samples, all the first PCR products (i.e. aliquots) from each panel of each sample are combined, thereby yielding a pool for each panel of each sample.

Thus in the case that multiple panels of analytes from the or each sample or detected in the detection assays, multiple aliquots are provided for each panel of the or each sample. That is to say, multiple aliquots are provided for the detection assay performed with each panel of proximity probe pairs.

The second PCR is performed separately on each pool in order to modify the reporter DNA molecules to prepare them for concatenation. This step is performed as described above. The second PCR is thus performed to provide defined end sequences to each reporter DNA molecule as described above, e.g. to provide assembly sequences for USER or Gibson assembly.

After the second PCR stage, the pools are combined and concatenation performed as described above. The concatemers may then be modified (as described above) and are then sequenced, as described above.

Alternatively viewed, the method described above may be defined as a method of detecting multiple analytes in one or more samples, wherein said analytes have varying levels of abundance in the sample(s), said method comprising:

performing a separate block of assays on each of separate multiple aliquots from the or each sample, to detect in each separate aliquot a subset of the analytes, wherein the analytes in each subset are selected based on their predicted abundance in the sample.

Each block of assays performed on an individual aliquot is, as detailed above, a multiplex assay (particularly a multiplex PEA). The multiplex assay to detect multiple analytes in the analyte subset (i.e. the analyte subset designated to be detected in any one particular aliquot) may thus be viewed as an “abundance block”. The term “abundance block” as used herein thus refers to a block of assays (or set of assays) performed to detect a particular group, or subset, of the analytes to be detected (i.e. assayed for) in a sample, wherein the analytes are assigned to each block (or set) of assays based on their abundance in the sample, namely their expected or predicted abundance, or relative abundance in the sample. In other words, the assays are grouped, or “blocked” based on abundance. Thus, different aliquots, or different abundance blocks, may be designated for the detection of a particular subset of analytes, based on, for example, low, high or varying degrees of intermediate levels of abundance etc. This does not imply that the abundance of each analyte in a block, or set of assays is the same or about the same; the abundance may vary between different analytes/assays in the block or set, and/or between different samples.

As mentioned above, this embodiment of the present method is for detecting multiple analytes in one or more samples, wherein the analytes have varying levels of abundance in the sample(s). That is to say, the analytes are present in the sample(s) at different concentrations, or at a range of concentrations. It is not required that every analyte in the or each sample is present at a substantially different concentration to every other analyte, but rather that not all analytes are present at substantially the same concentration. Although the analytes in the sample(s) are present at a range of concentrations, it may be that certain analytes are present at very similar concentrations.

It may be that the analytes are present in the sample(s) over a concentration range that spans several orders of magnitude. For instance, it may be that the analyte(s) present (or expected to be present) in the sample(s) at the highest concentration are present (or expected to be present) at a concentration about 1000-fold higher than the (expected) concentration of the analyte (expected to be) present at the lowest concentration in the sample(s). Analytes in a sample may, for instance, vary in concentration relative to each other about 10-fold, about 100-fold, about 1000-fold or more, and of course any value in between. In a clinical sample, analytes may be present across a range of several orders of magnitude, e.g. 3, 4, 5 or 6 or more orders of magnitude.

The level or value for the abundance which is used to block or group together different analytes, or more particularly the assays for different analytes, may not be dependent only on the absolute level or concentration of the analyte present in a sample (or expected to be present). Other factors may be considered, including the nature of the assay method, differences in performance of the assay for different analytes, etc. For example, in the case of detection assays based on antibodies or other binding agents, this may depend on antibody affinity for the analyte, or avidity etc. Such variability between assays for different analytes may be taken into account. For example the abundance may reflect the abundance of analyte that is detected in the assay, in terms of the assay output value or measurement. Accordingly, the predicted abundance on the basis of which analytes in a subset are selected may depend at least on the predicted level or concentration of the analyte in a sample, but it may also or alternatively depend on the predicted level of or value for abundance to be determined in a particular detection assay. Put another way, the abundance of an analyte in the sample may be its apparent abundance, or a notional abundance which depends on the detection assay. The apparent abundance of an analyte may vary depending on the assay used, and in particular the sensitivity of that assay.

The method comprises providing multiple (that is to say, at least two) aliquots from the, or each, sample. That is to say, multiple separate portions of the sample are provided. As noted above, multiple aliquots may be provided for each panel of assays for the, or each, sample. Each sample may be divided into multiple aliquots (such that the entire sample is aliquoted) or some of the, or each, sample may be provided as aliquots, without using the entire sample. The aliquots may be of the same size, or volume, or of different sizes, or volumes, or some aliquots may be of the same size and others of different sizes.

At least some of the aliquots may be diluted. For instance, aliquots may be diluted 1:2, 1:4, 1:5, 1:10, etc. In particular, aliquots may be subjected to 10-fold dilutions, i.e. one or more aliquots may be diluted 10-fold (or 1:10), one or more aliquots may be diluted 100-fold (1:100), and one or more aliquots may be diluted 1000-fold (1:1000). If desired, further dilutions may be made (e.g. 1:10,000 or 1:100,000), though as a rule a maximum dilution of 1:1000 can be expected to suffice. One or more aliquots may be undiluted (referred to herein as 1:1).

In a particular embodiment, a series of 10-fold dilutions is made, providing aliquots with the following dilutions: 1:1, 1:10, 1:100 and 1:1000. In this embodiment, the 1:10 dilution is generated by making a 10-fold dilution of the undiluted sample. The 1:100 and 1:1000 dilutions may be made by making direct 100-fold and 1000-fold dilutions (respectively) of the undiluted sample, or by making serial 10-fold dilutions of the 1:10 diluted aliquot (i.e. the 1:10 diluted aliquot may be diluted 10-fold to yield the 1:100 diluted aliquot, and the 1:100 diluted aliquot diluted 10-fold to yield the 1:1000 diluted aliquot). Sample dilutions (and indeed all pipetting steps throughout the methods of the invention) may be performed manually, or alternatively using an automated pipetting robot (such as an SPT Labtech Mosquito).

Dilutions of the aliquots may be made with any suitable diluent, which may depend on the type of sample being assayed. For instance, the diluent may be water or saline solution, or a buffer solution, in particular a buffer solution comprising a biologically-compatible buffer compound (i.e. a buffer compatible with the detection assay used, for instance a buffer compatible with a PEA or PLA). Examples of suitable buffer compounds include HEPES, Tris (i.e. Tris(hydroxymethyl)aminomethane), disodium phosphate, etc. Suitable buffers for use as diluent include PBS (phosphate-buffered saline), TBS (Tris-buffered saline), HBS (HEPES-buffered saline), etc. The buffer (or other diluent) used must be made up in a purified solvent (e.g. water) such that it does not contain contaminant analytes. The diluent should thus be sterile, and if water is used as diluent or the base of the diluent, the water used is preferably ultrapure (e.g. Milli-Q water).

Any suitable number of aliquots may be provided from the or each sample. As noted above, at least two aliquots are provided, though in most embodiments more than two will be provided. In a particular embodiment, as detailed above, four aliquots may be provided from each sample, or for each panel of assays from each sample: an undiluted sample aliquot and aliquots in which the sample is diluted 1:10, 1:100 and 1:1000. More or fewer aliquots than this may be provided, if more or fewer sample dilutions are desired. Moreover, one or more aliquots of each dilution factor may be provided, in accordance with the desires/requirements of the particular assay performed.

Once the multiple aliquots have been provided from the sample, a separate multiplex detection assay is performed for each aliquot (particularly a PEA), in order to detect a subset of the target analytes in each aliquot. A separate multiplex assay is performed for each aliquot, such that each aliquot is analysed separately (i.e. the multiple aliquots are not mixed during the multiplex reactions). Across all the aliquots provided from each sample, and upon which multiplex assays are performed, all the target analytes are detected. That is to say, across all the aliquots from each sample, assays are performed to determine whether each target analyte is present in or absent from the sample. However, each individual assay to detect a particular analyte may be performed in only one aliquot from each sample. Thus different subsets of analytes are detected in each aliquot from each sample, in other words different analytes are detected in each aliquot from a given sample. Preferably, the subsets detected in each aliquot from a particular sample are wholly different, i.e. each target analyte is detected in only one aliquot from each sample, such that there is no overlap between analyte subsets. However, in some embodiments particular analytes may be detected in multiple aliquots from each sample, if deemed appropriate. In this instance there would be some overlap of analytes between the subsets, in that some analytes would be present in multiple analyte subsets, but other analytes would be present in only one subset.

The analytes in each subset are selected based on their predicted abundance (i.e. concentration) in the sample or origin. That is to say, analytes which may be expected to be present in a sample at a similar concentration may be included in the same subset, and analysed in the same multiplex reaction. Conversely, analytes which may be expected to be present in a sample at different concentrations may be included in different subsets, and analysed in different multiplex reactions. Each analyte is assigned to a subset of analytes which are expected to be present at a similar concentration (e.g. a concentration within a particular order of magnitude) in the sample or origin. Each subset of analytes is then detected in an aliquot which is diluted by an appropriate factor in view of the expected concentrations of the analytes. Thus analytes expected to be present at the lowest concentrations may be detected in an undiluted aliquot, or an aliquot having a low dilution factor; analytes expected to be present at the highest concentrations are detected in the most diluted aliquot; and analytes expected to be present at concentrations in between these extremes are detected in aliquots having “in-between” dilution factors.

As noted above, in some embodiments certain analytes may be included in multiple subsets. This may for instance be the case if an analyte has an expected concentration essentially in between the expected concentrations of two subsets, such that it does not clearly “belong” to either of them. In this instance, the analyte may be included in both subsets. An analyte might also be included in two (or more) subsets if it is known that the analyte could be present in the sample or origin in an unusually wide range of concentrations.

It will be appreciated that given that the analytes in each subset are selected based on their predicted abundance in a sample, there may be different numbers of analytes in each subset. Alternatively there may be the same number of analytes in each subset, as appropriate.

The abundance/concentration of each analyte in a sample may be predicted based on known facts regarding the normal level of each analyte in the sample type to be analysed. For instance, if the sample is a plasma or serum sample (or a sample of any other bodily fluid), the concentration of the analytes therein may be predicted based on the known concentrations of species in these fluids. Normal plasma concentrations of a wide range of analytes of potential interest are available from www.olink.com/resources-support/document-download-center. However, as noted above, the abundance value used to allocate an analyte to a particular subset (block) can depend on the assay, and the results (e.g. measurements) which are obtainable from that assay.

As detailed above, the reporter DNA molecules generated in a PEA are amplified by PCR, and commonly the extension step that generates the reporter DNA molecules and the amplification step are performed within a single PCR. Particularly, when “abundance blocks” are used as described above to compensate for differences in analyte concentration in a sample, The PCR performed to amplify the reporter DNA molecules generated by the PEA (whether performed at the same time as generation of the reporter DNA molecules or separately) may be run to saturation. As is well known in the art, the amount of product of a PCR amplification relative to cycle number adopts the shape of an “5”. After a slow initial increase in amplicon concentration, a phase of exponential amplification is reached, during which the amount of product (approximately) doubles with each amplification cycle. Following the exponential phase a linear phase is reached, in which the amount of product increases in a linear, rather than exponential, fashion. Finally, a plateau is reached, in which the amount of product has reached its maximum possible level, given the reaction set-up and the concentration of components used, etc.

In the present method, a saturated PCR may be broadly considered to be any PCR which has moved beyond the exponential phase, i.e. a PCR in linear phase or that has plateaued. In a particular embodiment, “saturation” as used herein means that the reaction is run until the maximum possible product has been obtained, such that even if more amplification cycles are performed no more product is created (i.e. that the reaction is run until the amount of product plateaus). Saturation may be reached upon depletion of a reaction component, e.g. upon primer depletion or dNTP depletion. Depletion of a reaction component results in the reaction slowing and then entering a plateau. Less commonly, saturation may be reached upon polymerase exhaustion (i.e. if the polymerase loses its activity). Saturation may also be reached if the concentration of amplicon reaches such a high level that the concentration of DNA polymerase is not sufficient to maintain exponential amplification, i.e. if there are more amplicon molecules than polymerase molecules. In this instance, so long as ample primers and dNTPs remain in the reaction mix, the amplification enters and remains in linear phase.

A PCR amplification may be run to saturation simply by running it for a large number of cycles, such that saturation can be assumed. For instance, a PCR amplification run for at least 25, 30, 35 or more amplification cycles can be assumed to have reached saturation by the end point, in that the exponential amplification phase will have ended by that stage. Alternatively, saturation can be measured by quantitative PCR (qPCR). For instance, TaqMan PCR could be performed using a probe which binds a common sequence across all reporter DNA molecules, or qPCR could be performed using a dye which changes colour upon binding to double-stranded DNA, such as SYBR Green. The reaction can thus be followed and the minimum number of amplification cycles required to reach saturation determined. Either way, given that further processing of the amplified reporter DNA molecules is required (up to and including sequencing), it would be necessary to perform any such experimental qPCR to identify the point of saturation in a separate aliquot to that used experimentally to generate reporter DNA molecules for sequencing, since TaqMan probes or intercalating dyes are likely to interfere with the further steps of the method.

As detailed above, separate multiplex reactions are performed for each aliquot of the sample of interest. Each aliquot is used for detection of analytes present at different levels in the sample. Reporter DNA molecules will be initially generated in amounts corresponding to the amounts of each analyte in the sample. Thus for analytes present at high concentration, a high concentration of reporter DNA molecule can be expected to be generated; for analytes present at low concentration, a low concentration of reporter DNA molecule can be expected. It can be expected that the amount of reporter DNA molecule generated will be proportionate to the amount of corresponding analyte present in the sample, e.g. for a first analyte present in the sample at ten times the concentration of a second analyte, it can be expected that ten times as much reporter DNA molecule will be generated for the first analyte as for the second. Thus a much greater number of reporter DNA molecules will initially be generated in an aliquot used for detection of analytes expected to be present in the sample at high concentration than in an aliquot used for detection of analytes expected to be present in the sample at low concentration.

If this difference in reporter DNA molecule amount were carried through to the concatenation and sequencing steps, the reporter DNA molecules present in the highest amounts could “drown out” the reporter DNA molecules present in low amounts, resulting in poor detection of the analytes present in the sample in low amounts.

Amplification of the reporter DNA molecules from each multiplex reaction in a PCR run to saturation means that these differences in reporter DNA molecule concentration between aliquots will be removed. Once saturation has been reached essentially the same amount of reporter DNA molecule will be present in each aliquot. This means that similar amounts of reporter DNA molecule can be expected to be present for each analyte present in the sample, which in turn means that all reporter DNA molecules (and thus their corresponding analytes) should be detected when the reporter DNA molecules are concatenated and sequenced.

Running the first PCR to saturation is advantageous in the present method whether are not abundance blocks are used, because it ensures that each pool contains approximately the same number of reporter DNA molecules. As discussed above, that is advantageous as it ensures that the pooled reporter DNA molecules can be essentially exhausted during concatenation, rather than having a large proportion of reporter DNA molecules from one or more pools left over unconcatenated.

The methods described above enable the detection of each analyte of interest within a sample. The method also allows comparison of the levels of analytes within each subset for each sample, i.e. it allows comparison of the levels of analytes within each particular sample aliquot analysed. Within each individual aliquot, the levels of each different reporter DNA molecule generated are proportionate to the levels of their respective analytes (e.g. if a first analyte is present in a particular aliquot at twice the level of a second aliquot, twice as much reporter DNA molecule corresponding to the first analyte will be generated as reporter DNA molecule corresponding to the second analyte). This difference in levels of reporters will be detected during detection of the reporter DNA molecules, during sequencing, enabling comparison of the relative amounts of analytes present in a sample, but only for analytes detected in the same aliquot.

It is advantageous if the relative amounts of all analytes present in a sample can be compared (i.e. if comparison can be made between analytes detected in different aliquots). It is a further advantage if the relative amounts of analytes present in different samples can be compared. This can be achieved by including an internal control for each aliquot. The same internal control is included in each aliquot of each sample. The internal control is included in each aliquot of the sample at a different concentration, depending on the dilution factor of the aliquot. The concentration of the internal control is proportionate to the dilution factor of the aliquot. Thus, for instance, if the internal control is used at a particular given concentration in an undiluted sample aliquot, in a 1:10 diluted sample aliquot the internal control is used at a concentration one tenth of that used in the undiluted sample, and so on. This enables straightforward comparisons in relative concentrations of analytes between aliquots, while ensuring that the signal from the internal control does not overwhelm, and is not overwhelmed by, the signals from the analytes detected in the aliquots, as the internal control is present in each aliquot at a concentration appropriate for the analytes detected therein.

The internal control is, or results in the generation of, a control reporter DNA molecule. By comparing the amount of each reporter DNA molecule to the control reporter, the relative amounts of analytes analysed in different aliquots, and/or from different samples, can be compared. This is achievable because the relative difference between each reporter DNA molecule and the control reporter is comparable.

For instance, if two different reporter DNA molecules from different samples are present at the same relative level to the control reporter (e.g. 2- or 3-fold less or 2- or 3-fold more), this shows that the analytes indicated by the two reporter DNA molecules are present at essentially the same concentrations in the two samples. Similarly, if the ratio of a particular reporter DNA molecule to the control reporter is double that of the same reporter DNA molecule from a different sample to the control reporter (e.g. if the reporter molecule is present in the first sample at double the level of the control reporter, and the reporter molecule is present in the second sample at essentially the same level as the control reporter), this shows that the analyte indicated by the particular reporter DNA molecule is present in the first sample at approximately twice the level at which it is present in the second.

There are various alternatives which may be used as the internal control. Suitable controls may depend on the detection technique used. For any detection assay, the internal control may be a spiked analyte, i.e. a control analyte added to each aliquot at a defined concentration. The control analyte is added to the aliquot prior to the multiplex detection assay, and is detected in each aliquot in the same manner as the other analytes in the sample. In particular, detection of the control analyte leads to the generation of a control reporter DNA molecule, specific for the control analyte. If a control analyte is used, the control analyte is an analyte which cannot be present in the sample of interest. For instance, it may be an artificial analyte, or if the sample is derived from an animal (e.g. a human), the control analyte may be a biomolecule derived from a different species, which is not present in the animal of interest. In particular the control analyte may be a non-human protein. Exemplary control analytes include fluorescent proteins, such as green fluorescent protein (GFP), yellow fluorescent protein (YFP) and cyan fluorescent protein (CFP).

Another example of an internal control is a double-stranded DNA molecule having the same general structure as a reporter DNA molecule generated in the multiplex detection assay. That is to say, the DNA molecule comprises a barcode sequence which identifies it as a control reporter DNA molecule, and common primer binding sites, shared with all other reporter DNA molecules generated in response to analyte detection, to enable binding of the primers used in the amplification reaction(s). A double-stranded DNA molecule used as a control in this manner may be referred to as a detection control.

In a particular embodiment of the method, a control analyte and a detection control are both added to each aliquot. In this instance, clearly, the barcode sequence for the control analyte is different to the barcode sequence for the detection control, so that the two internal controls can be individually identified.

When a multiplex proximity extension assay is used for analyte detection, it is advantageous that an additional internal control is used: an extension control. The extension control is a single probe comprising an analyte-binding domain conjugated to a nucleic acid domain which comprises a duplex comprising a free 3′ end, which can be extended. In an embodiment, the extension control has a structure essentially equivalent to the duplex formed between two experimental probes upon their binding to their target analyte, except it comprises only a single analyte-binding domain. The analyte-binding domain used in the extension control does not recognise an analyte likely to be present in the sample of interest. A suitable analyte-binding domain is a commercially available, polyclonal isotype control antibody, such as goat IgG, mouse IgG, rabbit IgG, etc.

FIG. 2 shows examples of extension controls which can be used in the present method. Parts A-F correspond to extension controls which can be used in PEA assay Versions 1-6 of FIG. 1, respectively. The extension control is used to confirm that the extension step takes place as intended. Extension of the extension control yields a reporter DNA molecule which comprises a unique barcode, such that it may be identified as the extension control reporter nucleic acid molecule. When a multiplex PEA is used for analyte detection, it is advantageous that a control analyte, an extension control and a detection control are all used in the assay (e.g. are added to each aliquot). In other embodiments only two of the internal controls are used, e.g. a control analyte and an extension control, a control analyte and a detection control, or an extension control and a detection control.

Instead of a separate component of the PEA, the internal control may alternatively be a unique molecular identifier (UMI) sequence present in each reporter DNA molecule, which is unique to each molecule. By this is meant that each individual reporter DNA molecule generated during the initial stage of analyte detection comprises a UMI sequence.

Ordinarily when a PEA is performed multiple identical probe pairs for each analyte to be detected are applied to the sample. By “identical” probe pairs is meant that the multiple probe pairs all comprise the same pair of analyte-binding molecules, and the same pair of nucleic acid domains, such that every identical probe pair which binds a target analyte causes the generation of an identical reporter DNA molecule, which is indicative of the presence of that analyte in the sample.

When UMI sequences are utilised as the internal control, the probes used to detect each particular analyte are not identical. While a particular pair of analyte-binding molecules is used, each individual probe, or at least each individual probe comprising a particular one of the two analyte-binding molecules in the pair, comprises a different, unique nucleic acid domain. Each nucleic acid domain is rendered unique by the presence of a UMI sequence within it. This means that each specific pair of probes which binds to a particular analyte molecule leads to the generation of a unique reporter DNA molecule. A unique reporter DNA molecule is thus generated for every individual analyte molecule bound by a proximity probe pair. This allows for absolute quantification of the amount of the analyte present in the sample, since the precise number of analyte molecules detected can be counted based on the number of unique reporter nucleic acid molecules generated for that particular analyte.

Thus in a particular embodiment, the method comprises a step of performing multiple multiplex PEAs on one or more samples, each PEA yielding a pool of reporter DNA molecules, wherein each multiplex PEA comprises a PCR comprising an extension step that generates the reporter DNA molecules followed by an amplification step in which the reporter DNA molecules are amplified;

wherein an internal control is provided for each PCR, and said internal control is:

(i) a separate component which is present in a pre-determined amount, and which is, or comprises, or leads to the generation of, a control reporter DNA molecule which is amplified by the same primers as the reporter DNA molecules; or

(ii) a unique molecular identifier (UMI) sequence present in each reporter DNA molecule, which is unique to each molecule generated in the extension step.

The same one or more internal controls are used in each of the multiplex PEAs.

In a particular embodiment, the internal control (as described above) is, or comprises, or leads to the generation of, a control reporter DNA molecule wherein the control reporter DNA molecule comprises a sequence which is the reverse sequence of a reporter DNA molecule. That is to say that the control reporter DNA molecule comprises a sequence which is the reverse sequence of one of the reporter DNA molecules specific for an analyte being detected. It should be noted that “reverse” as used in this respect means precisely that, i.e. simply the reverse sequence, and not a reverse complement sequence. Since the control reporter DNA molecule has merely the reverse sequence of a reporter DNA molecule generated in response to detection of an analyte, the control reporter DNA molecule cannot hybridise to the reporter DNA molecule in question. This allows maintenance of a maximum level of similarity between the control reporter DNA molecule and the reverse sequence reporter DNA molecule generated in response to detection of an analyte, which is advantageous in PCR amplification, while avoiding unwanted hybridisation interactions between the control reporter DNA molecule and reporter DNA molecule generated in response to detection of an analyte. In particular, the control reporter DNA molecule may comprise a barcode sequence which is the reverse sequence of a barcode sequence of a reporter DNA molecule generated in response to detection of an analyte, but the same common universal sequences flanking the barcode as the reporter DNA molecules generated in the detection assay, to allow amplification of the control reporter DNA molecule along with the other reporter DNA molecules.

As mentioned above, in an embodiment, the detection assay used in the method uses a control analyte, an extension control and a detection control as internal controls. In order for these three controls to function together, it is apparent that the control reporter nucleic acid molecules generated/provided by the controls must be distinguishable from one another, i.e. must all have different sequences. In an embodiment, each control reporter DNA molecule used/generated has a sequence which is a reverse sequence of a reporter DNA molecule generated in response to detection of an analyte. In this case, clearly each control reporter DNA molecule has the reverse sequence of a different reporter DNA molecule generated in response to detection of an analyte.

Another challenge faced by proximity extension assays is that some “background” (i.e. false positive) signal is inevitable. Background signal may occur as a result of random interactions with or between unbound proximity probes in the reaction solution. Currently, the level of background signal in a proximity reaction is determined by the use of a separate negative control. For the negative control a proximity assay is performed using just buffer (i.e. no sample), such that all signal is background. Comparison of experimental assays to the negative control allows the true positive signal to be determined. This issue is addressed in co-pending application PCT/EP2021/058025, and the same methods used in that application may be utilised in the present application.

In particular, background control can be improved by using proximity probe pairs with shared hybridisation sites. This encourages the formation of “background” signal between all unbound probes sharing the same hybridisation sites. All signal from generated reporter DNA molecules is concatenated and read together (both true and false positive). True positive signal can be distinguished from false positive signal based on whether the reporter DNA molecule comprises paired barcode sequences (i.e. barcode sequences each corresponding to the same analyte, indicating a true positive signal) or unpaired barcode sequences (i.e. barcode sequences corresponding to different analytes, indicating a false positive signal). The level of false positive signal generated in the reaction indicates the level of background, meaning that a separate negative control reaction to determine background level no longer needs to be performed, simplifying the overall assay.

The use of shared hybridisation sites to determine background also mitigates against differences in the performance between different hybridisation sites. Different pairs of hybridisation sites may interact more or less strongly than others, resulting in different levels of background being produced from each pair of hybridisation sites. The shared hybridisation sites allow the level of background generated from each hybridisation site pair to be individually determined, resulting in a more accurate determination of the level of background to be calculated.

To this end, in one embodiment the proximity extension assay is performed by:

(i) contacting the or each sample (or aliquot thereof) with a plurality of pairs of proximity probes (as described above), wherein both probes within each pair comprise analyte-binding domains specific for the same analyte, and can simultaneously bind to the analyte; and each probe pair is specific for a different analyte;

wherein the nucleic acid domain of each proximity probe comprises a barcode sequence and a hybridisation sequence, wherein the barcode sequence of each proximity probe is different; and wherein:

in each proximity probe pair, the first proximity probe and the second proximity probe comprise paired hybridisation sequences, such that upon binding of the first and second proximity probe to their analyte, the respective paired hybridisation sequences of the first and second proximity probes hybridise to each directly or indirectly;

and wherein at least one pair of hybridisation sequences is shared by at least two pairs of proximity probes;

(ii) allowing the nucleic acid domains of the proximity probes to hybridise to one another, and performing an extension reaction as described above to generate a reporter DNA molecule comprising the barcode sequence of the first proximity probe and the barcode sequence of the second proximity probe; and

(iii) amplifying the reporter DNA molecule.

The reporter DNA molecules generated are processed, concatenated and sequenced as described above, and the relative amounts of each reporter DNA molecule determined. The analytes present in the or each sample are then identified, wherein in the identification step:
(a) reporter DNA molecules which comprise a first barcode sequence from a first proximity probe belonging to a first proximity probe pair and a second barcode sequence from a second proximity probe belonging to a second proximity probe pair are deemed background; and
(b) a reporter DNA molecule which comprises a first barcode sequence and a second barcode sequence from a proximity probe pair, and which is present in an amount higher than the background, indicates that the analyte specifically bound by the proximity probe pair is present in the sample.

As mentioned above, each sample (or aliquot thereof) is contacted with a plurality of pairs of proximity probes. Such a plurality of proximity probes may correspond to e.g. a panel of proximity probes as defined above, or a subset thereof. As noted above, each proximity probe comprises a unique barcode sequence (i.e. a different barcode sequence is present in each proximity probe). Notably, this does not mean that each individual probe molecule comprises a unique barcode sequence (though as noted above, each probe may comprise a UMI, in which case the UMI may or may not comprise or consist of the barcode sequence). Rather, each probe species comprises a unique barcode sequence. By “probe species” is meant a probe comprising a particular analyte-binding domain, and thus in other words, and as described for PEAs more generally above, all probe molecules comprising the same analyte-binding domain comprise the same unique barcode sequence. Every different probe species comprises a different barcode sequence.

As mentioned above, the nucleic acid domain of each proximity probe also comprises a hybridisation sequence. The hybridisation sequences are paired within each proximity probe pair. By “paired hybridisation sequences” is meant that the two hybridisation sequences within the pair are capable of directly or indirectly interacting with each other, such that when the method is performed and a pair of proximity probes bind to their target analyte, the nucleic acid domains of the two probes become directly or indirectly linked to one another.

In a particular embodiment, paired hybridisation sequences directly interact with each other, in which case they are complementary to one another, such that they hybridise to one another. In this embodiment, the hybridisation sequence of the first proximity probe in a pair is the reverse complement of the hybridisation sequence of the second proximity probe in the pair. This is the case in e.g. PEA Versions 1, 2, 4 and 6 of FIG. 1. In version 6, the hybridisation sites are the interacting sites of the two longer nucleic acid strand in the partially double-stranded nucleic acid domains (which as mentioned above may be referred to as splint oligonucleotides).

As described above, paired hybridisation sites may alternatively indirectly interact with each other. In this case, the paired hybridisation sequences do not hybridise directly to one another, but instead both hybridise to a separate, bridging oligonucleotide, i.e. a splint oligonucleotide. The separate oligonucleotide may be regarded as a third oligonucleotide in the assay method. In other words, in this case the paired hybridisation sequences are able to hybridise to a common oligonucleotide. This is the case in e.g. PEA Versions 3 and 5 of FIG. 1., which as described above utilise a splint oligonucleotide. In these embodiments, the paired hybridisation sites are the sites on the single-stranded probe nucleic acid domains which hybridise to the complementary sites on the splint.

When the paired hybridisation sequences interact indirectly, via a splint oligonucleotide, the splint oligonucleotide comprises two hybridisation sequences: one complementary to the hybridisation sequence of the first probe in the probe pair, and the other complementary to the hybridisation sequence of the second probe in the probe pair. The splint oligonucleotide is thus capable of hybridising to both of the paired hybridisation sequences of the proximity probes in its proximity assay set. Notably, the splint oligonucleotide is capable of hybridising to both of the paired hybridisation sequences of the proximity probes in its proximity assay set at the same time. Accordingly, when a pair of proximity probes bind their analyte and come into proximity, the nucleic acid domains of the probes both hybridise to the splint oligonucleotide, thus forming a complex comprising the two probe nucleic acid domains and the splint oligonucleotide.

In the present method, at least one pair of hybridisation sequences is shared by at least two pairs of proximity probes. In other words, at least two pairs of proximity probes (which bind to different analytes) have the same hybridisation sequences. Probes from pairs which share a pair of hybridisation sequences are capable of hybridising to each other, or forming a complex together. Hybridisation is most likely to occur between the nucleic acid domains of a pair of proximity probes when they are both bound to their respective analyte, since binding of the probes to the analyte brings the nucleic acid domains into close proximity. However, some interactions will inevitably form between paired hybridisation sequences of the nucleic acid domains of unbound proximity probes in solution (i.e. the nucleic acid domains of proximity probes which are not bound to their analyte), or when only one proximity probe has bound to its target analyte it may interact with another probe in solution. Notably, in solution the nucleic acid domain of an unbound proximity probe is equally likely to hybridise to (or form a complex with) the nucleic acid domain of any proximity probe which has a paired hybridisation sequence, regardless of whether the proximity probe binds the same analyte or a different analyte. Reporter DNA molecules generated as a result of such non-specific hybridisation (i.e. as a result of hybridisation between unbound proximity probes in solution) form background, as described further below.

In an embodiment, a significant proportion of probe pairs share their hybridisation sequences with at least one other proximity probe pair. In particular embodiments, at least 25%, 50% or 75% of proximity probe pairs share their hybridisation sequences with another proximity probe pair (i.e. with at least one other proximity probe pair). In a particular embodiment, all proximity probe pairs share their hybridisation sequences with at least one other proximity probe pair. However, as is apparent from the above, in another embodiment at least one pair of hybridisation sequences is unique to a single pair of proximity probes. That is to say, at least one pair of proximity probes does not share its hybridisation sequences with any other proximity probe pair. In particular embodiments, up to 75%, 50% or 25% of pairs of proximity probes do not share their hybridisation sequences with any other proximity probe pair.

In an embodiment, a single pair of hybridisation sequences is shared across all probe pairs which have shared hybridisation sequences. That is to say, all probe pairs which share their hybridisation sequences with another probe pair have the same pair of hybridisation sequences. In this embodiment, potentially all probe pairs used in the multiplex detection assay may have the same pair of hybridisation sequences.

However, if too many probe pairs share the same pair of hybridisation sequences, this can allow too large a number of background interactions to take place, hiding the true positive signals. Accordingly, it may be advantageous that each pair of hybridisation sequences is shared by a more limited number of probe pairs. In particular embodiments, no more than 20, 15, 10 or 5 proximity probe pairs share the same pair of hybridisation sequences. Thus it in an embodiment, the multiplex assay uses multiple sets of proximity probe pairs, each of which share a particular pair of hybridisation sequences. Thus all proximity probe pairs in a particular proximity probe pair set share the same pair of hybridisation sequences, but a different pair of hybridisation sequences is used by each different proximity probe pair set. This enables non-specific hybridisation between all probe pairs within each probe pair set, but prevents non-specific hybridisation between probe pairs in different probe pair sets. In general, each probe pair set comprises in the range 2 to 5 probe pairs, though larger sets may be used if preferred.

Once the reporter DNA molecules have been concatenated, detected by sequencing and counted, a determination step is performed, to determine which analytes are present in the sample. In this step, firstly the level of background is determined. All reporter DNA molecules generated as a result of non-specific probe interactions may be deemed background interactions. The relative amount of each of these background interactions is determined, such that the level of background interaction is determined. By “non-specific probe interactions” is meant interactions between probes which are not paired, i.e. interactions between probes which bind different analytes. Background reporter DNA molecules comprise a first barcode sequence from a first proximity probe belonging to a first proximity probe pair and a second barcode sequence from a second proximity probe belonging to a second proximity probe pair. Such reporter DNA molecules may alternatively by described comprising a first barcode sequence from a proximity probe specific for a first analyte and a second barcode sequence from a proximity probe specific for a second (or different) analyte. As described above, non-specific interactions between unpaired proximity probes may occur between probes free in solution, or when only one probe has bound to its analyte, as a result of their shared hybridisation sites.

Reporter DNA molecules generated by specific probe interactions are then analysed. By “specific probe interactions” is meant interactions between probes within a probe pair, i.e. between two probes which bind to the same analyte. Such reporter DNA molecules comprise a first barcode sequence and a second barcode sequence from a proximity probe pair. Such reporter DNA molecules may alternatively by described as comprising a first barcode sequence and a second barcode sequence from proximity probes specific for the same analyte.

Probes within a probe pair may also interact in solution, and so reporter DNA molecules generated by specific probe interactions may also constitute background (i.e. be generated as a result of background interactions). Therefore the amount of each reporter DNA molecules generated by specific probe interactions is compared to the level of background interaction, as determined by the amount of reporter DNA molecules generated as a result of non-specific probe interactions. If a reporter DNA molecule generated by a specific probe interaction is present at a higher level than the level of background interaction (i.e. the level of non-specific background reporter DNA molecules), this indicates that the analyte bound by the relevant probe pair is present in the sample. On the other hand, if a reporter DNA molecule generated by a specific probe interaction is present at a level which is no higher than the non-specific background reporter DNA molecules (e.g. if the reporter DNA molecule generated by a specific probe interaction is present at a level which is the same or lower than the non-specific background reporter DNA molecules), then the interaction between the relevant probe pair is deemed merely to be background. In this case, the fact that the interaction between the probes of the probe pair is merely background indicates that the analyte bound by the probe pair is not present in the sample.

Alternatively, for any individual target molecule, background interactions may be defined only as non-specific interactions including a probe which binds that target molecule. That is to say, for each target molecule background interactions may be defined as non-specific interactions between a probe which recognises the target molecule and an unpaired probe (i.e. a probe which does not recognise the target molecule) which shares its hybridisation site with the probe pair which recognises the target molecule. Thus in this case non-specific interactions between probes, neither of which recognise the target molecule, are not considered as background interactions for that particular target molecule.

In a particular embodiment, the level of background to which the level of a specific probe interaction is compared is the average level of the background interactions considered, in particular the mean level of the background interactions considered.

In a particular embodiment, the PEA further utilises one or more background probes which do not bind an analyte, said background probes comprising a nucleic acid domain comprising a barcode sequence and a hybridisation sequence shared with at least one proximity probe. “Background probes” may also be referred to herein as “inert probes”. As noted above the inert probes do not bind an analyte. Inert probes may nonetheless comprise an analyte-binding domain, if it is specific for an analyte which is known not to be present in the sample, in particular an antibody. The inert probe may in effect comprise a “binding domain” which is equivalent to the analyte-binding domain of a functional proximity probe but which does not perform an analyte-binding function, that is the binding domain equivalent is inert. In one embodiment, the inert domain may be provided by bulk IgG. Alternatively, inert probes may comprise an inactive analyte-binding domain, i.e. a non-functional analyte-binding domain. For instance, inert probes may comprise a sham analyte-binding domain, such as the constant region of an antibody, or one chain of an antibody (a heavy chain or a light chain only). Alternatively, inert probes may comprise an inert domain, to which the nucleic acid domain is attached but has no function and is not related to the analyte-binding domains of the active probes. An inert domain may be for example a protein which can be added to the assay without interfering with the assay reactions, such as serum albumin (e.g. human serum albumin or bovine serum albumin). In another alternative, the inert probes are simply nucleic acid molecules, and do not contain a non-nucleic acid domain.

Each inert probe comprises a barcode sequence within its nucleic acid domain. The inert probes each comprise a hybridisation sequence shared with at least one proximity probe. Preferably the inert probes each comprise a hybridisation sequence shared with multiple proximity probes. When inert probes are used, it may be that only a single species of inert probe is used, i.e. all inert probes have the same hybridisation sequence. Preferably however, multiple species of inert probe are used, each inert probe species comprising a different hybridisation sequences (shared with a different proximity probe or different group of proximity probes). It may be that each different species of inert probe has a different, unique, ID sequence. Alternatively, a common inert probe ID sequence may be used by all inert probes, of all different species. Either way, clearly the ID sequence or sequences used in the inert probes are not shared with any proximity probe.

Due to the hybridisation sites shared between the inert probes and certain proximity probes, background interaction in solution between inert probes and proximity probes is possible. Interaction of an inert probe with a proximity probe results in the formation of a reporter DNA molecule comprising the inert probe barcode sequence and the proximity probe barcode sequence. Reporter DNA molecules generated from interaction between an inert probe and a proximity probe are deemed background in the analyte identification step.

In a second aspect, the present disclosure and invention provides a kit, as detailed above. The kit is suitable for carrying out the method as defined and described herein, and comprises:

(i) multiple proximity probe pairs, wherein in each pair one proximity probe comprises a nucleic acid domain comprising a first universal primer binding site and a barcode sequence 3′ thereof, and the other proximity probe comprises a nucleic acid domain comprising a second universal primer binding site and a barcode sequence 3′ thereof;

(ii) a first primer pair, wherein the primers are designed to bind the first and second universal primer binding sites;

(iii) a set of assembly primer pairs suitable for preparing DNA molecules for directed assembly by USER assembly or Gibson assembly into a linear concatemer, wherein each primer comprises, from 5′ to 3′, an assembly site and a hybridisation site, and in each primer pair the hybridisation sites are designed to bind the first and second universal primer binding sites;

(iv) enzymes suitable for assembling DNA fragments by USER assembly or Gibson assembly, wherein the enzymes are suitable for use in the same means of DNA assembly as the assembly primer pairs; and

(v) a second primer pair, wherein each primer comprises a sequencing adaptor, a sequencing primer binding site, an index sequence and a hybridisation site, wherein the hybridisation sites are designed to bind the assembly sites of the assembly primers designed to form the ends of the linear concatemer;

and wherein the first primer in the pair comprises a first sequencing adaptor, a first sequencing primer site and a first index sequence, and the second primer in the pair comprises a second sequencing adaptor, a second sequencing primer site and a second index sequence.

The proximity probes and proximity probe pairs in the kit are as described above. In particular, the proximity probes are suitable for use in a proximity extension assay. In a particular embodiment, the proximity probes have the structure of the probes shown in PEA version 6 (FIG. 1), i.e. each probe comprises an analyte-binding domain conjugated to a partially single-stranded nucleic acid molecule. In each probe a short nucleic acid strand is conjugated to the analyte-binding domain, for example via its 5′ end. Each short nucleic acid strand is hybridised to a longer nucleic acid strand, which has a single-stranded overhang at its 3′ end (that is to say, the 3′ end of the longer nucleic acid strand extends beyond the 5′ end of the shorter strand conjugated to the analyte-binding domain). The overhangs of the two longer nucleic acid strands comprise hybridisation sites that are capable of hybridising to one another, forming a duplex.

In a particular embodiment, multiple pairs of proximity probes comprise nucleic acid domains that share a single pair of hybridisation sites, as described above.

In an embodiment, the assembly primer pairs and the enzymes are suitable for assembling DNA fragments by USER assembly. Thus the enzymes provided may be Uracil DNA glycosidase (UDG), DNA glycosylase-lyase endo VIII (EndoVIII) and DNA ligase. The assembly primers for preparing DNA molecules for USER assembly advantageously each comprise an assembly site comprising multiple uracil residues, as described above. In particular, each assembly site may comprise at least three uracil residues.

The second primer pair is as described above. As detailed above, in an embodiment each primer in the second primer pair comprises, from 5′ to 3′, the sequencing adaptor, the sequencing primer binding site, the index sequence and the hybridisation site. In an alternative embodiment each primer in the second primer pair may comprise, from 5′ to 3′, the sequencing adaptor, the index sequence, the sequencing primer binding site and the hybridisation site.

The kit may additionally comprise a DNA polymerase and a dNTP mix for performing one or more PCR steps. In particular the DNA polymerase may be suitable for performing PCR in the context of a PEA and/or USER assembly. The DNA polymerase may in particular be a Taq polymerase. The dNTP mix is a stock solution for PCR, and thus comprises the four standard dNTPs (dATP, dCTP, dGTP, dTTP).

The kit may also additionally comprise a buffer. The buffer is compatible with at least one enzyme provided in the kit. Preferably the buffer is compatible with both the assembly enzymes (e.g. USER enzymes) and the DNA polymerase, such that the buffer is, as described above, suitable for use in all stages of the method of the invention prior to sequencing.

The kit may also comprise one or more controls suitable for use in a PEA assay. The controls may be as described above, e.g. the kit may comprise a control analyte, an extension control and/or a detection control, as described above.

The methods and kits herein may be further understood by reference to the non-limiting examples below, and the figures.

### DESCRIPTION OF THE FIGURES

FIG. 1 shows a schematic representation of six different versions of proximity extension assays, described in detail above. The inverted ‘Y’ shapes represent antibodies, as an exemplary proximity probe analyte-binding domain.

FIG. 2 shows a schematic representation of examples of extension controls which may be used in proximity extension assays. Parts A-F show suitable extension controls for use in versions 1-6 of FIG. 1, respectively. In parts B-E, different possible extension controls for use in versions 2-5 of FIG. 1, respectively, are shown in options (i) and (ii). The legend for FIG. 1 also applies to FIG. 2.

FIG. 3 shows a comparison of normalised count number obtained by two PEA protocols, using 4 probe panels to assay a plasma sample. Normalised counts obtained using an “index inside” concatenation protocol are compared to normalised counts obtained using a method not including concatenation. A high correlation between the normalised counts obtained using the two protocols is seen (R=0.91).

FIG. 4 shows a comparison of normalised count number for IL-8 specifically from the assays compared in FIG. 3. A high correlation between the normalised counts obtained using the two protocols is seen for each panel (R=0.97-0.99).

FIG. 5 shows a comparison of normalised count number obtained by two PEA protocols, using 4 probe panels to assay a plasma sample. Normalised counts obtained using an “index inside” concatenation protocol are compared to normalised counts obtained using an “index outside” concatenation protocol. A high correlation between the normalised counts obtained using the two protocols is seen (R=0.98).

FIG. 6 shows a comparison of normalised count number for IL-8 specifically from the assays compared in FIG. 5. A high correlation between the normalised counts obtained using the two protocols is seen for each panel (R=0.99-1.00).

FIG. 7 shows a schematic representation of a method as disclosed herein, and depicts the generation of a concatemer comprising a PCR amplicon from each of 4 pools, A, B, C and D. Each pool comprises amplicons from a set of assays. PCR amplicons in each pool are generated by PCR1. A single amplicon from each pool is shown. In PCR2 the amplicons are provided with defined end sequences, which permit directed concatenation, using assembly primers. The assembly primers comprise a 5′ primer (“pool-specific” portion) which comprises the defined end sequence, and a 3′ primer hybridisation site (“universal” portion) which hybridises to the amplicon. A star (*) indicates a complementary sequence to the corresponding letter. For example, the sequence labelled “A*” is complementary to the sequence labelled “A.” The ends are digested. The digested products from pools A, B, C and D are pooled (combined), and ligated to generate a concatemeric product. PCR3 is performed to add sequencing adaptors to the ends.

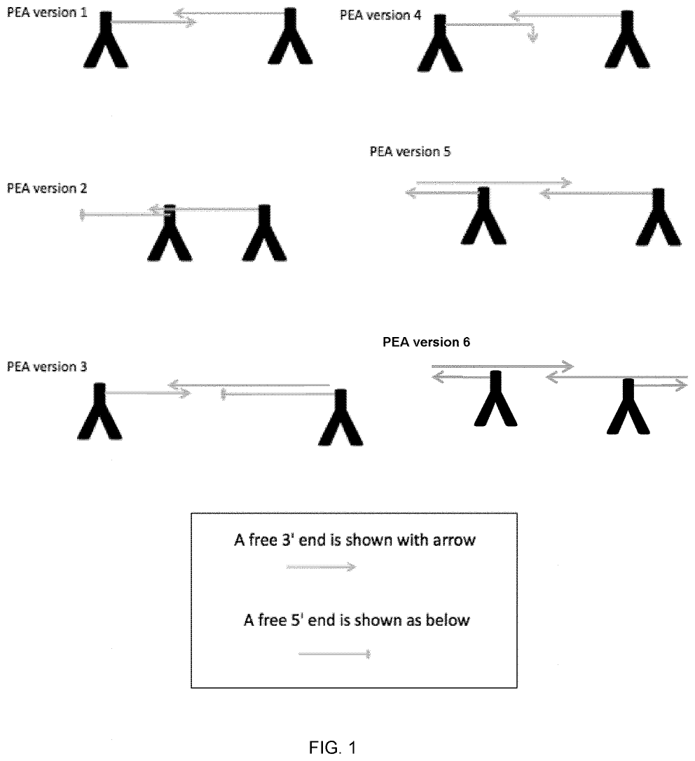

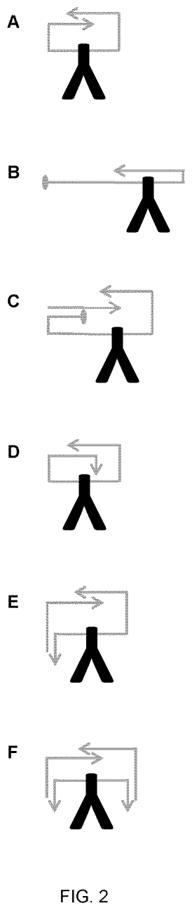

### EXAMPLES

### Example 1—Exemplary Experimental Protocol

### Step 1—Sample Preparation and Incubation

Sixteen aliquots from each of 48 to 96 plasma samples are incubated with one of each of 16 proximity probe sets (four abundance blocks from each of four 384-probe pair panels) in 96-well or 384-well incubation plates.
Samples may be pre-diluted 1:10, 1:100, 1:1000 and 1:2000 for those probe panels/groups containing assays that require it.
Dilution and dispensing of plasma samples into incubation solution can be performed manually, or by pipetting robot e.g. LabTech's Mosquito® HTS. Incubation solution is dispensed into the wells of the plate.
1 μl of sample is added to 3 μl of incubation mix at the bottom of each well, the plate is sealed with adhesive film, spun at 400×g for 1 minute at room temperature and incubated overnight at 4° C.
If using the above-mentioned pipetting robot, volumes may be decreased to 0.2 μl sample and 0.6 μl incubation mix (5× reduction).
   The tables below give exemplary reagent formulations. Other components may be included, for example other blocking agents in the probe solutions.

TABLE 1
Sample Diluent and Negative
Control Solution

| Component | Concentration | |
|---|---|---|
| NaCl | 8.01 | g/l |
| KCl | 0.2 | g/l |
| Na₂HPO₄ | 1.44 | g/l |
| KH₂PO₄ | 0.2 | g/l |
| BSA | 1 | g/l |

TABLE 2
Incubation Mix

| | 4 μl | 0.8 μl |
|---|---|---|
| | Incubation | Incubation |
| | Volume | Volume |
| Reagent | Volume (μl) | Volume (μl) |
| Incubation Solution | 2.40 | 0.48 |
| Forward Probe Solution | 0.30 | 0.06 |
| Reverse Probe Solution | 0.30 | 0.06 |
| Sample | 1.00 | 0.20 |
| Total | 4.0 | 0.8 |

TABLE 3
Incubation Solution

| Component | Concentration | |
|---|---|---|
| Triton X-100 | 1.70 | g/l |
| NaCl | 8.01 | g/l |
| KCl | 0.2 | g/l |
| Na₂HPO₄ | 1.44 | g/l |
| KH₂PO₄ | 0.2 | g/l |
| EDTA Na-salt | 1.24 | g/l |
| BSA | 8.80 | g/l |
| Blocking-probes Mix | 0.199 | g/l |
| GFP | 1-5 | pM |

TABLE 4
Forward Probe Solution

| Component | Concentration | |
|---|---|---|
| NaCl | 8.01 | g/l |
| KCl | 0.2 | g/l |
| Na₂HPO₄ | 1.44 | g/l |
| KH₂PO₄ | 0.2 | g/l |
| EDTA Na-salt | 1.24 | g/l |
| Triton X-100 | 1 | g/l |
| BSA | 1 | g/l |
| Probes | 1-100 | nM per probe |

TABLE 5
Reverse Probe Solution

| Component | Concentration | |
|---|---|---|
| NaCl | 8.01 | g/l |
| KCl | 0.2 | g/l |
| Na₂HPO₄ | 1.44 | g/l |
| KH₂PO₄ | 0.2 | g/l |
| EDTA Na-salt | 1.24 | g/l |
| Triton X-100 | 1 | g/l |
| BSA | 1 | g/l |
| Probes | 1-100 | nM per probe |
| Detection Control | 6.4-1188 | fM |
| Extension Control | 75-10686 | fM |

### Step 2—Proximity Extension and Reporter Molecule Amplification

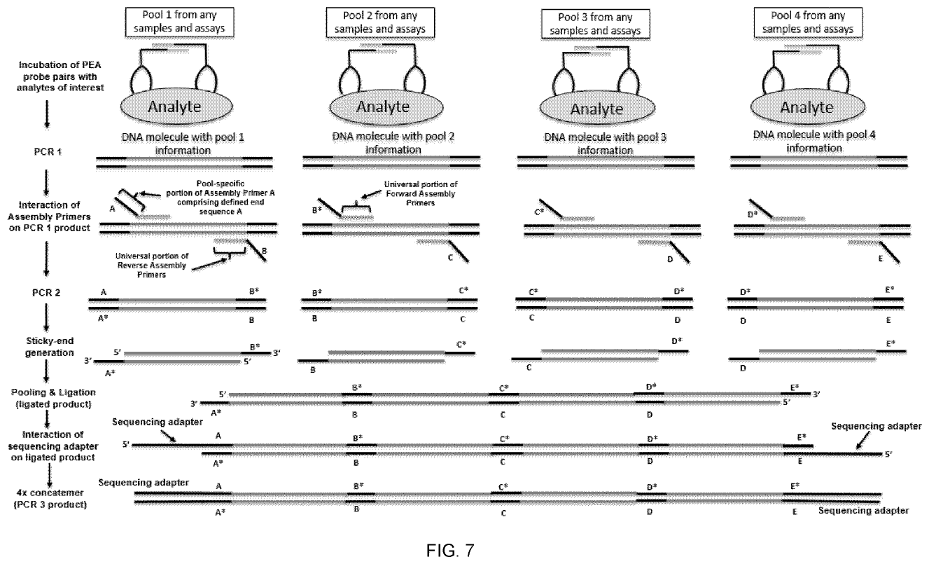

Extension and amplification are performed using Pwo DNA polymerase. The PCR is performed using common primers for amplification of all extension products. (See, for example, PCR1 in FIG. 7)

The incubation plate (from step 1) is brought to room temperature and centrifuged at 400×g for 1 minute. The extension mix (comprising ultrapure water, DMSO, Pwo DNA polymerase and reaction solution) is added to the plate, and the plate is then sealed, briefly vortexed and centrifuged at 400×g for 1 minute, then placed in a thermal cycler for the PEA reaction and amplification (50° C. 20 min, 95° C. 5 min, (95° C. 30 s, 54° C. 1 min, 60° C. 1 min)×25 cycles, 10° C. hold). Preferably, a dispensing robot may be used to dispense the extension mix into the plate, e.g. the Thermo Scientific™ Multidrop™ Combi Reagent Dispenser.

TABLE 6
PEA PCR Reaction Mix

| | 4 μl | 0.8 μl |
|---|---|---|
| | Incubation Volume | Incubation Volume |
| Reagent | Volume (μl) | Volume (μl) |
| MilliQ water | 75.0 | 15.00 |
| DMSO (100%) | 10.0 | 2.00 |
| Reaction Solution | 10.0 | 2.0 |
| DNA Polymerase | 1.0 | 0.2 |
| (1-10 U/μ1) | | |
| Incubation mix | 4.0 | 0.8 |
| Total | 100.0 | 20.0 |

TABLE 7
Reaction Solution

| Component | Concentration | |
|---|---|---|
| Tris base | 168.40 | mM |
| Tris-HCl | 31.47 | mM |
| MgCl₂ hexahydrate | 10.00 | mM |
| dATP | 2.00 | mM |
| dCTP | 2.00 | mM |
| dGTP | 2.00 | mM |
| dTTP | 2.00 | mM |
| Forward primer | 10.00 | μM |
| Reverse primer | 10.00 | μM |

### Step 3—Pooling Abundance Blocks

PCR products from each of the abundance blocks from each 384-probe pair panel from each sample are pooled together. This results in four mixtures (pools) of PCR products per sample, one for each 384-probe pair panel. Each pool in this case is thus a mixture, or collection, of PCR products which corresponds to a panel of proximity probes, or in other words, a panel of assays performed on a sample. The pool is made up of the PCR products derived from four abundance blocks (i.e. there are four abundance blocks for each panel. Each block corresponds to a set of assays, based on the relative abundances of the analytes under test in each assay).

Different volumes can be taken from each abundance block to even out the relative numbers of assays between the blocks. Pooling of PCR products can be performed manually, or by pipetting robot.

Step 4—Amplification with Assembly Primers

For each mixture of PCR products (i.e. the product of each 384-probe pair panel) from each sample, a separate second PCR is performed using assembly primers for USER assembly. This is depicted as PCR2 in FIG. 7. Each assembly primer comprises a “pool-specific” portion, which comprises or provides the defined end sequence to be added to the amplicon and a “universal” portion that hybridises to the amplicon; the universal portion, and its complementary binding site, are shared between the amplicons of different pools. A set of USER assembly primers is used for the various panel products of each sample. An exemplary set of assembly primers is shown in the table below (as shown, each primer has a unique assembly site, which with the exception of the terminal assembly sites have a neighbouring complementary site, and each of the forward and reverse hybridisation sites are, respectively, the same). One pair of assembly primers is used for amplification of the products of each panel (which corresponds to each pool) from a sample, e.g. using the exemplified primers, for each sample Pair A is used for panel 1, Pair B for panel 2, Pair C for panel 3 and Pair D for panel 4 (corresponding to pools 1-4 as depicted in FIG. 7). The products of the first PCR are added to a second PCR mix (comprising Taq polymerase, dNTPs, universal buffer and assembly primers in ultrapure water) and PCR is performed: 95° C. 3 min, (95° C. 30 sec, 45° C. 30 sec, 72° C. 1 min)×5 cycles, (95° C. 30 sec, 65° C. 30 sec, 72° C. 1 min)×10 cycles, 10° C. hold.

TABLE 8
Second PCR Mix

| Reagent | Volume | |
|---|---|---|
| Polymerase Buffer (20X stock) | 0.5 | μl |
| dNTPs (25 mM of each) | 0.08 | μl |
| Taq polymerase (5 U/μl) | 0.05 | μl |
| MilliQ Water | 4.87 | μl |
| Assembly Primers (5 μM of each) | 2.5 | μl |
| PEA-PCR Product (0.1 μM) | 2 | μl |
| Total Volume: | 10 | μl |

TABLE 9
Assembly Primers

| | | |
|---|---|---|
| Pair A Forward | 5′ CCUCUGCUGCUCUCAUUGUCGCTCTTCCGATCT 3′ | SEQ ID NO: 5 |
| Pair A Reverse | 5′ ACACUGUACGUTAGAGACTCCAAGC 3′ | SEQ ID NO: 6 |
| Pair B Forward | 5′ ACGUACAGUGUCGCTCTTCCGATCT 3′ | SEQ ID NO: 7 |
| Pair B Reverse | 5′ AGCUCAAUCCUTAGAGACTCCAAGC 3′ | SEQ ID NO: 8 |
| Pair C Forward | 5′ AGGAUUGAGCUCGCTCTTCCGATCT 3′ | SEQ ID NO: 9 |
| Pair C Reverse | 5′ ACAGACUUACUTAGAGACTCCAAGC 3′ | SEQ ID NO: 10 |
| Pair D Forward | 5′ AGUAAGUCUGUCGCTCTTCCGATCT 3′ | SEQ ID NO: 11 |
| Pair D Reverse | 5′ GUGCGUGCAUGAUCCUACUTAGAGACTCCAAGC 3′ | SEQ ID NO: 12 |

Assembly sites are underlined.
Uracil residues for USER assembly are highlighted in bold.

### Step 5—Digestion

The products of Step 4 are digested to degrade the uracil-containing assembly sites, leaving 3′ overhangs at the end of each PCR product. The product of each separate second PCR is digested separately. The second PCR products are added to USER enzymes and incubated at 37° C. for 60 to 120 minutes.

TABLE 9
Digestion Mix

| Reagent | Volume | |
|---|---|---|
| Enzyme Buffer (20X) | 1 | μl |
| Endo VIII (10 U/μl) | 1 | μl |
| UDG (1 U/μl) | 1 | μl |
| Second PCR Product (1.25 μM) | 10 | μl |
| Total Volume: | 13 | μl |

### Step 6—Concatenation

The digested products of each PEA panel (each panel representing a pool of products from four abundance blocks) from each sample are combined and ligated to generate a concatemer comprising a product from each panel of the sample in question. The products are concatenated in the order defined by the complementary overhangs generated from the assembly sites. In the example above, where Panel 1 was amplified with assembly primer pair A, Panel 2 with assembly primer pair B, Panel 3 with assembly primer pair C and Panel 4 with assembly primer pair D, the products of the panels are concatenated in the order Panel 1-Panel 2-Panel 3-Panel 4.

TABLE 10
Ligation Mix

| Reagent | Volume | |
|---|---|---|
| ATP (10 mM) | 1 | μl |
| T4 Ligase (400 U/μl) | 1 | μl |
| Pooled Digested Product (240 nM) | 8 | μl |
| Total Volume: | 10 | μl |

### Step 7—Attachment of Sequencing Adaptors

For Illumina sequencing, sequencing adaptors are added to both ends of each concatemer. This is performed in a third PCR (depicted as PCR3 in FIG. 7), which is also used to add sequencing primer binding sites and index sequences to identify the sample from which each concatemer derives. The primers for the third PCR comprise, from 5′ to 3′, a sequencing adaptor (e.g. the P5 and P7 adaptors, mentioned above), a sequencing primer binding site (e.g. Rd1SP and Rd2SP binding sites, mentioned above), an index sequence and the hybridisation site.

Ligated concatemers are added to a third PCR mix comprising Taq polymerase, primers, buffer and dNTPs, and amplified: 95° C. 3 min, (95° C. 30 sec, 60° C. 30 sec, 72° C. 1 min)×5 cycles, (95° C. 30 sec, 65° C. 30 sec, 72° C. 1 min)×15 cycles, 10° C. hold.

TABLE 11
Third PCR Mix

| Reagent | Volume | |
|---|---|---|
| MilliQ Water | 5.5 | μl |
| Polymerase Buffer (20X) | 1 | μl |
| dNTP Mix (2.5 mM of each) | 0.8 | μl |
| Taq Polymerase (5 U/μl) | 0.05 | μl |
| Forward Primer (100 μM) | 0.1 | μl |
| Reverse Primer (100 μM) | 0.1 | μl |
| Ligation Product (1.92 nM) | 2 | μl |
| Total Volume: | 10 | μl |

### Step 8—Sequencing

Concatemers are pooled and then sequenced using an Illumina platform (e.g. the NoveSeq platform). By generating concatemers comprising reporter DNA molecules from four panels, the throughput of each sequencing run is increased four-fold.

### Step 9—Data Output

Barcode (from each reporter DNA molecule) and index (from each concatemer) sequences are identified in the data, counted, summed and aligned/labeled according to a known barcode-assay-sample key.
“Matching barcodes” represent interactions between two paired PEA probes. The count is relative to the number of interactions in the PEA.
Counts for each assay and sample must be normalised using the internal reference controls to be able to compare between samples.
Each abundance block has its own internal reference control.

### Example 2—Reference Example of Method without Concatenation

This reference protocol is disclosed in co-pending application PCT/EP2021/058008. In this protocol, steps 1 to 3 were performed as in Example 1. Thereafter the protocol was as follows:

### Step 4—PCR2 Indexing

A primer plate containing 48 to 96 reverse primers is provided (generally one primer in each well of a 96-well plate). Each reverse primer comprises the “IIlumina P7” sequencing adapter sequence (SEQ ID NO: 2) and a sample index barcode. A unique barcode sequence is used for PCR1 products (i.e. the products of the PCR performed in Step 2) from each different sample. Preferably each of the up to four PCR1 pools comprising the same plasma sample (one for each 384-probe pair panel) receive the same index sequence, for easy identification and data processing. A forward common primer comprising the “Illumina P5” sequencing adapter sequence (the same forward primer as used in PCR1) is provided in the PCR2 solution.

Each PCR1 pool is contacted with PCR2 solution containing the forward common primer, a single reverse (index) primer from the primer plate, and a DNA polymerase (Taq or Pwo DNA polymerase). Amplification is performed by PCR until primer depletion (95° C. 3 min, (95° C. 30 s, 68° C. 1 min)×10 cycles, 10° C. hold).

The theoretical end concentration of pooled PCR1 product is 1 μM (all primers used). PCR1 amplicons are diluted 1:20 dilution for PCR2, giving a starting concentration of 50 nM in each PCR2 reaction. The concentration of each PCR2 primer is 500 nM. PCR2 primer depletion should therefore occur after 3.3 cycles (10-fold amplification).

TABLE 8
PCR2 Reaction Mix

| Reagent | Volume (μl) |
|---|---|
| MilliQ water | 14.96 |
| PCR2 solution | 2.0 |
| DNA Polymerase (1-10 U/μ1) | 0.04 |
| Sample index primer solution | 2.0 |
| Pooled PCR1 reactions | 1.0 |
| Total | 20.0 |

TABLE 9
PCR2 Solution

| Component | Concentration | |
|---|---|---|
| Tris base | 168.40 | mM |
| Tris-HCl | 31.47 | mM |
| MgC1₂ hexahydrate | 10.00 | mM |
| dATP | 2.00 | mM |
| dCTP | 2.00 | mM |
| dGTP | 2.00 | mM |
| dTTP | 2.00 | mM |
| Forward “P5” Primer | 5.00 | μM |

TABLE 10
Index Primer Solution

| Component | Concentration | |
|---|---|---|
| Tris base | 1.948 | mM |
| Tris-HCl | 8.052 | mM |
| EDTA | 1 | mM |
| Index “P7” primer | 5.00 | μM |

### Step 5—End Pool

All 48 to 96 indexed sample pools belonging to the same 384-probe pair panel are pooled together, adding the same volume from each sample. This yields up to four final pools (or libraries), one for each 384-probe pair panel.

### Step 6—Purification and Quantification (Optional)

The libraries are purified separately using magnetic beads, and purified libraries' total DNA concentration is determined using qPCR with a DNA standard curve. AMPure XP beads (Beckman Coulter, USA), which preferentially bind longer DNA fragments, may be used in accordance with the manufacturer's protocol. The AMPure XP beads bind the long PCR products but do not bind short primers, thus enabling purification of the PCR product from any remaining primers.

Depletion of the PCR2 primers means that this purification step may not be necessary.

### Step 7—Quality Control (Optional)

A small aliquot of each (purified) library is analysed on an Agilent Bioanalyser (Agilent, USA), in accordance with the manufacturer's instructions, to confirm successful DNA amplification.

### Step 8—Sequencing

Libraries are sequenced using an Illumina platform (e.g. the NoveSeq platform). Each of the up to four libraries (from each 384-probe pair panel) is run in a separate “lane” of a flow cell. Depending on the size and model of flow cell and sequencer used, the up to four libraries may be sequenced in parallel or sequentially (one after the other) in different flow cells.

### Step 9—Data Output

Barcode (from each reporter nucleic acid molecule) and sample index (from the sample index primers) sequences are identified in the data, counted, summed and aligned/labeled according to a known barcode-assay-sample key.
“Matching barcodes” represent interactions between two paired PEA probes. The count is relative to the number of interactions in the PEA.
Counts for each assay and sample must be normalised using the internal reference controls to be able to compare between samples.
Each of the four abundance blocks has its own internal reference control.
   Each 384-probe pair panel is separated based on the lane it is read out in. Each panel comprises the same 96 sample indexes and the same 384 barcode combinations and internal reference controls.

### Example 3—Sequencing of Concatenated and Unconcatenated Reporters

Three reaction protocols were compared:

1. A protocol as described above in Example 1 (referred to as “Index Inside”).

2. A protocol as described above in Example 1, with the exception of a difference in the primers used for the third PCR. In protocol 2, the primers for the third PCR were arranged differently to in Example 1. Specifically, the primers for the third PCR comprised, from 5′ to 3′, a sequencing adaptor, an index sequence, a sequencing primer binding site and the hybridisation site (i.e. the order of the index sequence and the sequencing primer binding site is reversed, referred to as “Index Outside”).

3. A protocol as described in Example 2.

For each of the three protocols, eight plasma samples were tested and compared. Each sample was assayed using four panels of PEA probes, each of which contained 372 probe pairs. Each of the panels included a probe pair for detection of IL-8. After sequencing, all matched barcode reads (counts) within each abundance block were normalized against an internal control. The normalised barcode counts generated by each protocol were compared.

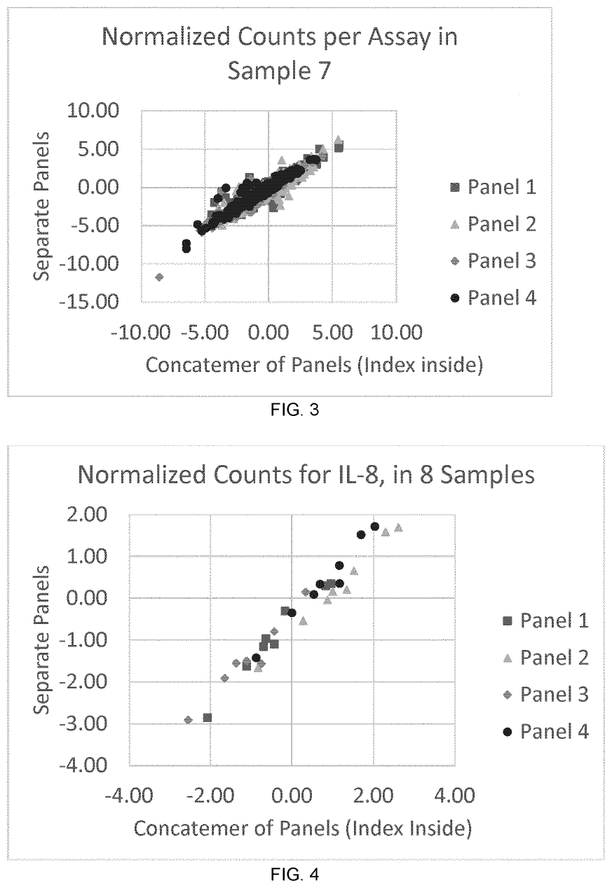

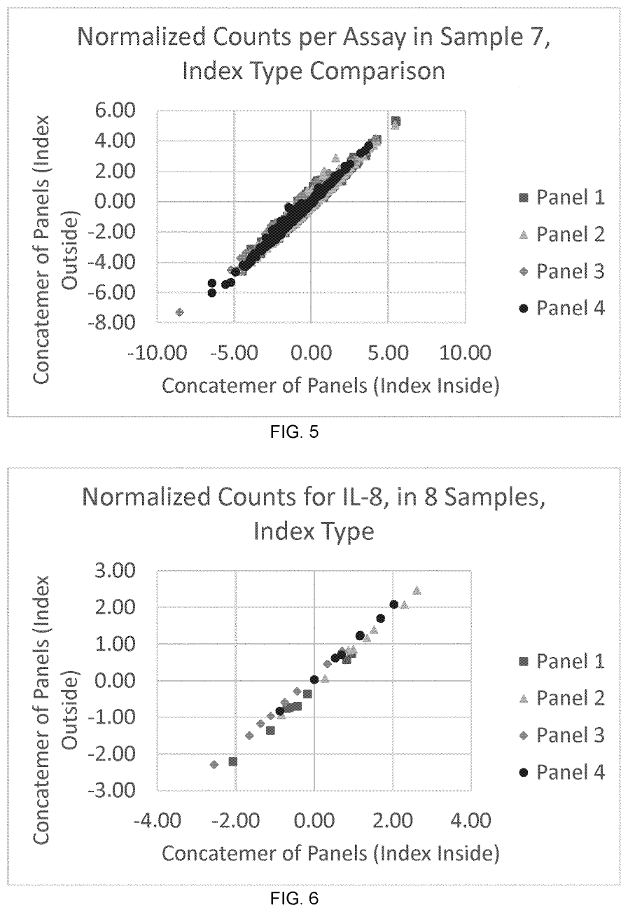

A comparison of the normalised counts obtained from protocols 1 and 3 for one sample (sample 7) is shown in FIG. 3. The figure shows a high correlation (R²=0.91) between the normalised counts obtained with the two different protocols (and similar R² values were obtained for the other seven samples as well), showing that the two different protocols generate approximately the same number of normalised barcode counts for each probe pair used to assay the sample. The normalised counts obtained from protocols 1 and 2 for the same sample were also compared, as shown in FIG. 5. The figure shows a very high correlation (R²=0.98) between the normalised counts obtained with the two different protocols (and similar R² values were obtained for the other 7 samples as well), showing that there is essentially no difference between the performance of the “index inside” and “index outside” protocols.

The normalised counts from the different protocols for IL-8 were also specifically compared. The counts for IL-8 obtained from each assay panel using protocols 1 and 3 for each of the 8 samples were compared, as shown in FIG. 4. The figure shows a high level of correlation between the normalised counts obtained with the two methods (R² values between 0.97 and 0.99 for the four different assay panels). The same comparison was made for normalised counts obtained using protocols 1 and 2, as shown in FIG. 6. The figure shows a very high level of correlation between the normalised counts obtained with the two methods (R² values between 0.99 and 1 for the four different assay panels).

These results show that very similar results are obtained when assaying a sample using a PEA method comprising a concatenation step as provided herein, as when using the earlier method in which each reporter DNA molecule is individually sequenced. If a sample contains a high or low level of a particular target protein (e.g. IL-8), this is correctly identified in all of the three protocols tested. As detailed above, concatenation allows a significant improvement in throughput of the method, and these results show that the improvement in throughput is obtained without any loss of accuracy.

What is claimed is:

1. A method of detecting DNA sequences from multiple pools, wherein each pool comprises at least one species of DNA molecule, the method comprising:
(i) combining the pools to form a combination pool;
(ii) in the combination pool, generating at least one linear DNA concatemer containing one DNA molecule from each pool, wherein a position of each DNA molecule within the concatemer correlates to the pool from which the DNA molecule originated; and
(iii) sequencing the concatemers, thereby detecting the DNA sequence of each DNA molecule at each position in each concatemer, wherein each detected DNA sequence is assigned to the pool from which its DNA molecule originated based upon its position within the concatemer.

2. The method of claim 1, wherein the method comprises, prior to step (i), in each pool, joining to each DNA molecule of the pool a first end sequence, and, when the number N of multiple pools is greater than two, for at least N-2 pools, joining to each DNA molecule of each N-2 pool, a second end sequence, wherein each end sequence is different from the other end sequences and each end sequence of each pool is configured to join to one end sequence in one other pool to form the linear DNA concatemers.

3. The method of claim 1, wherein each DNA molecule is an amplicon generated in a DNA amplification reaction.

4. The method of claim 1, wherein each DNA molecule is a reporter DNA molecule specific for an analyte, and sequencing of each reporter DNA molecule results in detection of the corresponding analyte.

5. The method of claim 4, wherein the reporter DNA molecules are generated by a multiplex detection assay performed on a sample; and
the method comprises performing multiple multiplex detection assays on one or more samples, in order to detect multiple analytes in each sample, and each multiplex detection assay yields a pool of reporter DNA molecules.

6. The method of claim 5, wherein each multiplex detection assay comprises a first PCR which generates a respective first PCR product;
and wherein the first PCR products are modified by a second PCR, in order to prepare the first PCR products for concatenation, wherein the second PCR generates the multiple pools of DNA molecules.

7. The method of claim 5, wherein the detection assay is a proximity extension assay, comprising an extension step that generates the reporter DNA molecules, and an amplification step in which the reporter DNA molecules are amplified, and the extension and amplification steps take place within a single PCR.

8. The method of claim 7, wherein the multiple multiplex proximity extension assays are performed on the same sample; and
wherein each proximity extension assay comprises detecting analytes using pairs of proximity probes, each proximity probe comprising:
(i) an analyte-binding domain specific for an analyte; and
(ii) a nucleic acid domain,
wherein both probes within each pair comprise analyte-binding domains specific for the same analyte, and each probe pair is specific for a different analyte, and wherein each probe pair is designed such that on proximal binding of the pair of proximity probes to their respective analyte the nucleic acid domains of the proximity probes interact to generate a reporter DNA molecule;
wherein at least 2 panels of proximity probe pairs are used, each panel being for the detection of a different group of analytes, and each multiplex proximity extension assay uses one panel of proximity probe pairs;
wherein (a) within each panel, every probe pair comprises a different pair of nucleic acid domains; and (b) in different panels the probe pairs comprise the same pairs of nucleic acid domains; and
wherein the product of each panel of proximity probe pairs forms one of the multiple pools.

9. The method of claim 1, wherein concatenation is performed by USER assembly or Gibson assembly.

10. The method of claim 9, wherein the method comprises performing a PCR on each pool using assembly primers, wherein all the DNA molecules in one pool are amplified using the same primer pair, and a different primer pair is used for amplification in each pool, and wherein each primer of the primer pairs comprises a unique assembly site which is complementary to one unique assembly site in one other pool; and
wherein in step (ii), the PCR products of each pool are joined to the PCR products of different pools via their complementary assembly sites, thereby generating the linear concatemers.

11. The method of claim 10, wherein concatenation is performed by USER assembly, and each assembly site comprises multiple uracil residues.

12. The method of claim 10, wherein:
(a) each DNA molecule is a reporter DNA molecule specific for an analyte and obtained by performing multiple multiplex proximity extension assays, the multiple multiplex proximity extension assays generating the multiple pools of reporter DNA molecules, wherein the reporter DNA molecules in each pool comprise universal primer binding sites at their 3′ and 5′ termini;
(b) the linear concatemers are formed by USER assembly comprising:
(i) processing the PCR products in each pool to generate 3′ overhangs comprising the assembly sites;
(ii) combining the pools; and (iii) generating the multiple linear DNA concatemers, the PCR products of each pool being joined to the PCR products of different pools having complementary 3′ overhangs; and
(d) sequencing the concatemers, thereby identifying the analytes detected in each proximity extension assay; wherein the analytes detected in each proximity extension assay are identified based on the combination of the sequence of each reporter DNA molecule and its position within its concatemer.

13. The method of claim 1, wherein the linear DNA concatemers are subjected to a PCR to add at least a first sequencing adaptor to the concatemers.

14. The method of claim 13, wherein in the PCR a first sequencing adaptor is added to one end of the concatemers, and a second sequencing adaptor is added to the other end of the concatemers.

15. The method of claim 1, wherein the linear DNA concatemers are subjected to a PCR to add at least a first sequencing primer binding site to the concatemers.

16. The method of claim 15, wherein in the PCR a first sequencing primer binding site is added at one end of the concatemers, and a second sequencing primer binding site is added at the other end of the concatemers.

17. The method of claim 1, wherein:
(I) multiple sets of pools are individually combined and a separate concatenation reaction performed for each set of pools, yielding multiple concatenation reaction products;
(II) a unique index sequence is added to each concatenation reaction product by PCR;
(III) the concatenation reaction products are combined; and
(IV) the concatemers are sequenced, and the index sequence identifies the set of pools from which each concatemer originates.

18. The method of claim 17, wherein in the PCR a first index sequence is added at one end of the concatemers, and a second index sequence is added at the other end of the concatemers.

19. The method of claim 18, wherein the concatemers are subjected to a single PCR, in which a sequencing adaptor, a sequencing primer binding site, and an index sequence are added to both ends of each concatemer.

20. The method of claim 19, wherein the PCR to which the concatemers are subjected yields products comprising, at each end, from 5′ to 3′, a sequencing adaptor, a sequencing primer binding site, and an index sequence.

21. A method of detecting multiple analytes in one or more samples, comprising:
(i) performing multiple multiplex detection assays on one or more samples, in order to detect multiple analytes in each sample, wherein each multiplex detection assay is a proximity extension assay comprising an extension step that generates reporter DNA molecules, and an amplification step in which the reporter DNA molecules are amplified, wherein the extension and amplification steps take place within a single PCR and yield a pool of amplified reporter DNA molecules, each reporter DNA molecule being specific for an analyte,
(ii) performing a PCR on each pool using assembly primers, wherein all the reporter DNA molecules in one pool are amplified using the same primer pair, and a different primer pair is used for amplification in each pool, and wherein each primer of the primer pairs comprises a unique assembly site which is complementary to one unique assembly site in one other pool;
(iii) combining the PCR products of each pool to form a combination pool;
(iv) in the combination pool, forming by USER assembly linear DNA concatemers containing a PCR product of one reporter DNA molecule from each pool, wherein a position of each PCR product of a reporter DNA molecule within the concatemer correlates to the pool from which the reporter DNA molecule originated;
(v) subjecting the concatemers to a single PCR in which a sequencing adaptor, a sequencing primer binding site, and an index sequence are added to both ends of each concatemer; and
(vi) sequencing the concatemers, thereby identifying the analytes detected in each proximity extension assay based on the combination of the sequence of each reporter DNA molecule and its position within its concatemer.

22. A kit comprising:
(i) multiple proximity probe pairs, wherein each proximity probe comprises:
an analyte-binding domain specific for an analyte; and
a nucleic acid domain,
wherein in each pair, the nucleic acid domain of one proximity probe comprises a first universal primer binding site and a barcode sequence 3′ thereof, and the nucleic acid domain of the other proximity probe comprises a second universal primer binding site and a barcode sequence 3′ thereof, wherein both probes within each pair comprise analyte-binding domains specific for the same analyte, and each probe pair is specific for a different analyte, and wherein each probe pair is designed such that on proximal binding of the pair of proximity probes to their respective analyte the nucleic acid domains of the proximity probes interact to generate a reporter DNA molecule;
(ii) a first primer pair, wherein the primers are designed to bind the first and second universal primer binding sites;
(iii) a set of assembly primer pairs suitable for preparing DNA molecules for directed assembly by USER assembly or Gibson assembly into a linear concatemer, wherein each primer comprises, from 5′ to 3′, an assembly site and a hybridisation site, and in each primer pair the hybridisation sites are designed to bind the first and second universal primer binding sites;
(iv) enzymes suitable for assembling DNA fragments by USER assembly or Gibson assembly, wherein the enzymes are suitable for use in the same means of DNA assembly as the assembly primer pairs; and
(v) a second primer pair, wherein each primer comprises a sequencing adaptor, a sequencing primer binding site, an index sequence and a hybridisation site, wherein the hybridisation sites are designed to bind the assembly sites of the assembly primers designed to form the ends of the linear concatemer;
and wherein the first primer in the pair comprises a first sequencing adaptor, a first sequencing primer binding site and a first index sequence, and the second primer in the pair comprises a second sequencing adaptor, a second sequencing primer binding site and a second index sequence.
